SEQUENCE LISTING (42 sequences)

SEQ ID NO: 1 - protein, 7 aa, Artificial Sequence
  synthetic lipogenic adenovirus fiber protein peptide for antibody production
FNPVYPY

SEQ ID NO: 2 - protein, 12 aa, Artificial Sequence
  synthetic lipogenic adenovirus fiber protein peptide for antibody production
NIPFLTPPFVSS

SEQ ID NO: 3 - protein, 15 aa, Artificial Sequence
  synthetic lipogenic adenovirus fiber protein peptide for antibody production
FPPGVLSLKLADPIA

SEQ ID NO: 4 - protein, 17 aa, Artificial Sequence
  synthetic lipogenic adenovirus fiber protein peptide for antibody production
ANGNVSLKVGGGLTVEQ

SEQ ID NO: 5 - protein, 14 aa, Artificial Sequence
  synthetic lipogenic adenovirus fiber protein peptide for antibody production
SGKLSVDTKAPLQV

SEQ ID NO: 6 - protein, 9 aa, Artificial Sequence
  synthetic lipogenic adenovirus fiber protein peptide for antibody production
AGHGLAVVT

SEQ ID NO: 7 - protein, 13 aa, Artificial Sequence
  synthetic lipogenic adenovirus fiber protein peptide for antibody production
SLPSLVGTLVVLT

SEQ ID NO: 8 - protein, 7 aa, Artificial Sequence
  synthetic lipogenic adenovirus fiber protein peptide for antibody production
PSPNCKV

SEQ ID NO: 9 - protein, 32 aa, Artificial Sequence
  synthetic lipogenic adenovirus fiber protein peptide for antibody production
SKLTLALTKCGSQILATVSLLVVTGKYAIISD

SEQ ID NO: 10 - protein, 20 aa, Artificial Sequence
  synthetic lipogenic adenovirus fiber protein peptide for antibody production
NPKQFSIKLLFNDKGVLLSD

SEQ ID NO: 11 - protein, 7 aa, Artificial Sequence
  synthetic lipogenic adenovirus fiber protein peptide for antibody production
YKEAVGF

SEQ ID NO: 12 - protein, 14 aa, Artificial Sequence
  synthetic lipogenic adenovirus fiber protein peptide for antibody production
LGGEVYQPGFIVVK

SEQ ID NO: 13 - protein, 7 aa, Artificial Sequence
  synthetic lipogenic adenovirus fiber protein peptide for antibody production
ANCAYSI

SEQ ID NO: 14 - protein, 12 aa, Artificial Sequence
  synthetic lipogenic adenovirus fiber protein peptide for antibody production
WGKVYKDPIPYD

SEQ ID NO: 15 - protein, 10 aa, Artificial Sequence
  synthetic lipogenic adenovirus fiber protein peptide for antibody production
VETARDSKLT

SEQ ID NO: 16 - protein, 14 aa, Artificial Sequence
  synthetic lipogenic adenovirus fiber protein peptide for antibody production
LGGEVYQPGFIVVK

SEQ ID NO: 17 - protein, 12 aa, Artificial Sequence
  synthetic lipogenic adenovirus fiber protein peptide for antibody production
WGKVYKDPIPYD

SEQ ID NO: 18 - protein, 8 aa, Artificial Sequence
  synthetic lipogenic adenovirus fiber protein peptide for antibody production
GTGSSAHG

SEQ ID NO: 19 - DNA, 20 bp, Artificial Sequence
  synthetic PCR amplification Forward primer for Ad-36 hexon protein
ggtggacaaccacccactac

SEQ ID NO: 20 - DNA, 20 bp, Artificial Sequence
  synthetic PCR amplification Forward primer for Ad-36 hexon protein
tggacaaccacccactacaa

SEQ ID NO: 21 - DNA, 20 bp, Artificial Sequence
  synthetic PCR amplification Reverse primer for Ad-36 hexon protein
cagcggagcttgtatcttcc

SEQ ID NO: 22 - DNA, 23 bp, Artificial Sequence
  synthetic nested PCR outer forward primer for Ad-36 fiber protein unique region
gtctggaaaactgagtgtggata

SEQ ID NO: 23 - DNA, 24 bp, Artificial Sequence
  synthetic nested PCR outer reverse primer for Ad-36 fiber protein unique region
atccaaaatcaaatgtaatagagt

SEQ ID NO: 24 - DNA, 22 bp, Artificial Sequence
  synthetic nested PCR inner forward primer for Ad-36 fiber protein unique region
ttaactggaaaaggaataggta

SEQ ID NO: 25 - DNA, 24 bp, Artificial Sequence
  synthetic nested PCR inner reverse primer for Ad-36 fiber protein unique region
ggtgttgttggttggcttaggata

SEQ ID NO: 26 - DNA, 25 bp, Artificial Sequence
  synthetic probe for Ad-36 fiber coat protein
agttgaaacagcaagagactcaaag

SEQ ID NO: 27 - DNA, 25 bp, Artificial Sequence
  synthetic probe for Ad-36 fiber coat protein
ggtactggatcaagtgcacatggag

SEQ ID NO: 28 - DNA, 28 bp, Artificial Sequence
  synthetic probe for Ad-36 fiber coat protein
ttgaaacagcaagagactcaaagctaac

SEQ ID NO: 29 - DNA, 20 bp, Artificial Sequence
  synthetic Forward primer for detecting Ad-36 fiber protein using SYBR GREEN method
taagccaaccaacaacacca

SEQ ID NO: 30 - DNA, 20 bp, Artificial Sequence
  synthetic Reverse primer for detecting Ad-36 fiber protein using SYBR GREEN method
tgcacaattggcatcagttt

SEQ ID NO: 31 - DNA, 20 bp, Artificial Sequence
  synthetic Forward primer for detecting Ad-36 fiber protein using SYBR GREEN method
ggccatggtttagcagttgt

SEQ ID NO: 32 - DNA, 20 bp, Artificial Sequence
  synthetic Reverse primer for detecting Ad-36 fiber protein using SYBR GREEN method
gtccaaagggtgcgtgtatc

SEQ ID NO: 33 - DNA, 22 bp, Artificial Sequence
  synthetic Forward primer for detecting Ad-36 fiber protein using SYBR GREEN method
ttaactggaaaaggaataggta

SEQ ID NO: 34 - DNA, 24 bp, Artificial Sequence
  synthetic Reverse primer for detecting Ad-36 fiber protein using SYBR GREEN method
ggtgttgttggttggcttaggata

SEQ ID NO: 35 - DNA, 24 bp, Artificial Sequence
  synthetic Forward primer for detecting Ad-36 fiber protein using Taqman method
caatgggaatgtctcactcaaggt

SEQ ID NO: 36 - DNA, 22 bp, Artificial Sequence
  synthetic Reverse primer for detecting Ad-36 fiber protein using Taqman method
agggtgccttagtatccacact

SEQ ID NO: 37 - DNA, 22 bp, Artificial Sequence
  synthetic Reverse primer for detecting Ad-36 fiber protein using Taqman method
cagttttccagactgttgttct

SEQ ID NO: 38 - DNA, 1089 bp, human adenovirus type 36
  human lipogenic adenovirus Ad36 fiber protein
atgtcaaagaggctccgggtggaagatgacttcaaccccgtctacccctatggctacgcgcggaatcagaatatccccttcctcactcccccctttgtctcctccgatggattccaaaacttcccccctggggtcctgtcactcaaactggctgatccatgtctcactcaaggtgggagggggactcactgtagaacaacagtctggaaaactgagtgtggatactaaggcacccttgcaagttgcaaatgacaacaaattggagctatcttatgatgatccatttaaggtagagaataacaaacttggaattaaagctggccatggtttagcagttgtaactaaagaaaacacaagtcttcctagtctagttggaacacttgtagttttaactggaaaaggaataggtactggatcaagtgcacatggaggaactattgatgtaagacttggtgaaggaggtgggtatcatttgatgaaaaaggagacttagtagcttgggacaaaaaaaatgatacacgcaccctttggacaacacctgatccttctccaaattgcaaagttgaaacagcaagagactcaaagctaaccttagcacttacaaaatgtggtagtcaaattttggccactgtatctttacttgttgttacgggcaaatatgctattataagtgacacagtcaacccaaagcagttctctattaagttactgtttaatgacaagggtgttttgttaagtgactcaaatcttgatgggacatattggaactatagaagcaacaataacaacataggcactccttataaagaggctgttggttttatgccaagcacaacagcttatcctaagccaaccaacaacaccagcacagatccggataaaaaagtgagtcaaggtaaaaataaaattgtaagcaatatatcttggaggagaggtatatcaaccaggatttattgttgttaaatttaatcaggaaactgatgccaattgtgcatactctattacatttgatttggatggggtaaggtgtataaggatcctataccatatgatacctcttctactttctcatatatcgctcaagaatga

SEQ ID NO: 39 - protein, 371 aa, human adenovirus type 36
  human lipogenic adenovirus Ad36 fiber protein
MSKRLRVEDDFNPVYPYGYARNQNIPFLTPPFVSSDGFQNFPPGVLSLKLADPIAIANGNVSLKVGGGLTVEQQSGKLSVDTKAPLQVANDNKLELSYDDPFKVENNKLGIKAGHGLAVVTKENTSLPSLVGTLVVLTGKGIGTGSSAHGGTIDVRLGEGGGLSFDEKGDLVAWDKKNDTRTLWTTPDPSPNCKVETARDSKLTLALTKCGSQILATVSLLVVTGKYAIISDTVNPKQFSIKLLFNDKGVLLSDSNLDGTYWNYRSNNNNIGTPYKEAVGFMPSTTAYPKPTNNTSTDPDKKVSQGKNKIVSNIYLGGEVYQPGFIVVKFNQETDANCAYSITFDFGWGKVYKDPIPYDTSSFTFSYIAQE

SEQ ID NO: 40 - DNA, 2775 bp, human adenovirus type 36
  human lipogenic adenovirus Ad36 hexon protein, major capsid protein
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccatgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagtttggcccccaagggcgcccccaactccagtcagtggactgacaaagaacggcaaaatggtggacaaccacccactacaaaagatgttacaaaaacattcggagtagcagccaggggagggcttcatattactgataaaggactacaaataggagaagatgaaaataacgaggatggtgaagaagagatatatgcagacaaaactttccagccagaacctcaagtaggagaggaaaactggcaagatactgatgttttctatggcggcagagcgcttaaaaaggaaaccaaaatgaaaccatgctatggctcttttgccagacctaccaatgaaaaaggaggtcaagctaaatttttaaatggcgaaaacggtcaaccttctaaagatcaagatattacattagctttctttgatcttaaacaaaatgacactggaactactcaaaaccagccagatgttgtcatgtacactgaaaatgtgtatctggaaaccccagacacccatgtggtgtacaaacctggcaaggaagatacaagctccgctgctaaccttacacaacagtccatgcccaacaggcccaactacattggtttcagggacaactttgtggggctcatgtattacaacagcactggcaacatgggtgtgctggctggtcaggcctctcagttgaatgctgtggttgacttgcaagacagaaacaccgagctgtcatatcagctcttgctagattctctgggtgacagaaccagatactttagcatgtggaattctacggtggacagctatgatccagatgtcaggatcattgagaatcacggtgttgaagatgagcttccaaattattgcttcccactggatggatctggcagcaataccgcatatcaaggtgttaaatatgaaaacggagctggcaatggaagctggaaagtagatggcgaagttgcttctcagaatcagatcgccaagggtaatctgtatgccatggagataaaccttcaggccaacctgtggaagagttttctgtactcgaacgtggcgctgtatctaccagactcctacaagtacacgccggccaacatcacgctgcccaccaacaccaacacctacgagtacatgaacggccgcgtggtggcaccctcgctggtggatgcctatgtcaacatcggtgcccgctggtcgctggaccccatggacaacgtcaaccccttcaaccaccaccgcaacgcgggtctgcgctaccgctccatgcttctgggcaacggccgctacgtgcccttccacatccaagtgccccaaaagttctttgccatcaagaacctgctcctgcttcccggttcctacacctacgagtggaacttccgcaaggatgtcaacatgatcctgcaaagttccctcggcaacgacctgcgcgtcgacggcgcctccgtccgcttcgacagcgtcaacctctatgccaccttattccccatggcgcgcaacaccgcctccacccttgaagccatgctgcgcaacgacaccaacgaccagtccttcaacgactacctctcggccgccaacatgctctacccaatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccattccctacctcgacggaaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgactaaggactggttcctcgtccagatgctctctcattacaacattggctaccagggcttctacgtgcctgagggttacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccataccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgaacgccaccacataa

SEQ ID NO: 41 - protein, 944 aa, human adenovirus type 36
  human lipogenic adenovirus Ad36 hexon protein, major capsid protein
MATPSMMPQWAYMHIAGQDASEYLSPGLVQFARATDTYFSLGNKFRNPTVAPTHDVTTDRSQRLTLRFVPVDREDTTYSYKARFTLAVGDNRVLDMASTYFDIRGVLDRGPSFKPYSGTAYNSLAPKGAPNSSQWTDKERQNGGQPPTTKDVTKTFGVAARGGLHITDKGLQIGEDENNEDGEEEIYADKTFQPEPQVGEENWQDTDVFYGGRALKKETKMKPCYGSFARPTNEKGGQAKFLNGENGQPSKDQDITLAFFDLKQNDTGTTQNQPDVVMYTENVYLETPDTHVVYKPGKEDTSSAANLTQQSMPNRPNYIGFRDNFVGLMYYNSTGNMGVLAGQASQLNAVVDLQDRNTELSYQLLLDSLGDRTRYFSMWNSTVDSYDPDVRIIENHGVEDELPNYCFPLDGSGSNTAYQGVKYENGAGNGSWKVDGEVASQNQIAKGNLYAMEINLQANLWKSFLYSNVALYLPDSYKYTPANITLPTNTNTYEYMNGRVVAPSLVDAYVNIGARWSLDPMDNVNPFNHHRNAGLRYRSMLLGNGRYVPFHIQVPQKFFAIKNLLLLPGSYTYEWNFRKDVNMILQSSLGNDLRVDGASVRFDSVNLYATLFPMARNTASTLEAMLRNDTNDQSFNDYLSAANMLYPIPAKATNVPISIPSRNWAAFRGWSFTRLKTKETPSLGSGFDPYFVYSGSIPYLDGTFYLNHTFKKVSIMFDSSVSWPGNDRLLTPNEFEIKRSVDGEGYNVAQCNMTKDWFLVQMLSHYNIGYQGFYVPEGYKDRMYSFFRNFQPMSRQVVDEINYKDYKAVTLPFQHNNSGFTGYLAPTMRQGQPYPANFPYPLIGQTAVPSVTQKKFLCDRVMWRIPFSSNFMSMGALTDLGQNMLYANSAHALDMTFEVDPMDEPTLLYLLFEVFDVVRVHQPHRGVIEAVYLRTPFSAGNATT

SEQ ID NO: 42 - DNA, 35155 bp, human adenovirus type 36
  human adenovirus type 36 genomic sequence
catcatcaataaatataccccacaaagtaaacaaaagttaatatgcaaatgagcttttgaatttggggcgtggccgacgctgattggacgagaagtggtgatgcaaatgacgtcacgacgcacggccgacggtcgccgcggaggcgtggcctagtccggaagcaagtcgcgrggctgatgacgtataaaaaagcggactttagacccggaaacggccgattttcccgcggtcacgcccggatatgaggtaattctgggcggatgcaagtgaaattaggccattttggcgcgaaaactgaatgaggaagtgaaaagtgaaaaataccgggcccgcccagggcggaatatttaccgagggccgagagactttgaccgattacgtgggggtttcgattgcggtgtttttttcgcgaatttccgcgtccgtgtcaaagtccggtgtttatgtcacagatcagctgatccacagggtatttaaaccagtcgagcccgtcaagaggccactcttgagtgccagcgagtagagatttctctgagctccgctcccagagaccgagaaaaatgagacacctgcgccttctaccttcaactgtgcccggcgacctggctgtgattatgctggaggactttgtgaatacagttctggaggacgaactgcatccagagccatttgagctgggacctacacttcaggacctctatgatctggaggtagatgcccatgatgacgaccctaacgaagaggctgtgaatttaatatttccagaatctatgattcttcaggctgacatagccagtgaagccatagttactcctctacatactcccactctgcctcccatacctgaattggaggaggatgaagaaatagacctccggtgctacgaggaaggttttcctcccagcgattcagaggacgaacagggtgagcagcagatggctctaatctctgatttagcttgtgtgattgtggaggaacaagttgtgattgaaaaatctaccgagccagtacaaggctgtaggaactgccagtatcaccgggataagtccggagacccgaacgcttcctgcgctctgtgttacatgaaatctactttcagctttatttacagtaagtggagtgaatgtgagagaggctgagtgcttaacacataactgtaatgcttgaacagctgtgctaagtgtggtttatttttgttactaggtccggtgtcagaggatgagtcatcaccctcagaagaagaccacccgtctccccctgagctgtcaggcgaaacgcccctgcaagtgcacagacccaccccagtcagagccagtggcgagaggcgagcagctgtagaaaaaattgaggacttgttacatgacatgggtggggatgaacctttggacctgagcttgaaacgccccaggaactaggcgcagctgcgcttagtcatgtgtaaataaagttgtacaataaaagtatatgtgacgcatgcaaggtgtggtttatgactcatgggcggggcttagtcctatataagtggcaacacctgggcacttgggcacagaccttcagggagttcctgatggatgtgtggactatccttgcagactttagcaagacacgccggcttgtagaggatagttcagacgggtgctccgggttctggagacactggtttggaactcctctatctcgcctggtgtatacagttaagaaggattataaagaggaatttgaaaatctttttgctgactgctctggtctgctagattctctgaatcttggccaccagtcccttttccaggaaagggtactccacagccttgatttttccagcccagggcgcactacagccggggttgcttttgtggtttttctggttgacaaatggagccaggacacccaactgagcaggggctacatcctggacttcgcagccatgcacctgtggagggcctggatcaggcagcggggacagagaatcttgaactactggcttctacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcaaggtgctgacatccatggccaggggagtgaagagggagaggagcgatgggggtaataccgggatgatgaccgagctgactgccagtctgatgaatcggaagcgcccagagcgccttacctggtacgagctacagcaggagtgcagggatgagataggcctgatgcaggataaatatggcctggagcagataaaaacccattggttgaacccagatgaggattgggaggaggctattaagaagtatgccaagatagccctgcgcccagattgcaagtacatagtgaccaagaccgtgaatatcagacatgcctgctacatctcggggaacggggcagaggtggtcatcgataccctggacaaggccgccttcaggtgttgcatgatgggaatgagagcaggagtgatgaatatgaattccatgatcttcatgaacattaagttcaatggagagaagtttaatggggtgctgttcatggccaacagccacatgaccctgcatggctgcagcttcttcggtttcaacaacatgtgcgccgaggtctggggagctgctaagatcaggggatgtaagttttatggctgctggatgggcgtggtcggaagacccaagagcgagatgtctgtgaagcagtgtgtgtttgagaaatgctacctgggagtctctaccgagggcaatgctagagtgagacactgctcttccatggagacgggctgcttctgcctggtgaagggcacggcctctctgaagcataatatggtgaagggctgcacggatgagcgcatgtacaacatgctgacctgcgattcgggggtctgccatatcctgaagaacatccatgtgacctcccaccccagaaagaagtggccagtgtttgagaataacctgctgatcaagtgccatatgcacctgggtgccagaaggggcaccttccagccgtaccagtgcaactttagccagaccaagctgctgttggagaacgatgccttctccagggtgaacctgaacggcatctttgacatggatgtctcggtgtacaagatcctgagatacgatgagaccaagtccagggtgcgcgcttgcgagtgcgggggcagacacaccaggatgcaaccagtggccctggatgtgacagaggagctgagaccagaccacctggtgatggcctgtaccgggaccgagttcagctccagtggggaggacacagattagaggtaggttttgagtagtgggcgtggctaaggtgagtataaaggcggtgtcttacgagggtctttttgcttttctgcagacatcatgaacgggaccggcggggccttcgaaggggggctttttagcccttatttgacaacccgcctgccgggatgggccggagttcgtcagaatgtgatgggatctacggtggatgggcgcccagtgcttccagcaaattcctcgaccatgacctacgcgaccgtggggagctcgtcgctcgacagcaccgccgcagccgcggcagccgcagccgccatgacagcgacgagactggcctcgagctacatgcccagcagcagcagtagcccctctgtgcccagttccatcatcgccgaggagaaactgctggccctgctggcagagctggaagccctgagccgccagctggccgccctgacccagcaggtgtccgagctccgcgagcaacagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgggggtggaggtagcaccactgcatggcctcgtgctctggggtcgtgttgtagatgatccagtcatagcaggggcgctgggcgtggtgctggatgatgtccttaaggaggagactgatggccacggggagccccttggtgtaggtgttggcgaagcggttgagctgggagggatgcatgcggggggagatgatgtgcagtttggcctggatcttgaggttggcaatgttgccgcccagatcccgcctggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcgtaattatgctcctgggtgagatcatcataagacattttaatgaatttggggcggagggtgccagattgggggacgatagttccctcgggccccggggcaaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcatgtctctgacttggaggttttcccggacgagctcgccgaggaggcggtccccgcccagcgagaggagctcttgcagggaagcaaagtttttcaggggcttgagtccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagtgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagagaagggattgcagggcgtagagcttgggtgcgagaaaaacggactcgggggcgaaggcgtccgctccgcagtgggcgcagacggtctcgcactcgacgagccaggtgagctcgggctgctcggggtcaaaaaccagttttcccccgttctttttgatgcgcttcttacctcgcgtctccatgagtctgtgtccgcgctcggtgacaaacaggctgtcggtgtccccgtagacggacttgatgggcctgtcctgcagggacgtcccgcggtcctcctcgtagagaaactcggaccactctgagacaaaggcgcgcgtccacgccaagacaaaggaggccacgtgcgaggggtagcggtcgttgtccaccagggggtccaccttttccaccgtgtgcagacacatgtccccctcctccgcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctctagagcgggcatgacctcggcactcaggttgtcagtttctagaaacgaggaggatttgatgttggcctgccctgccgcgatgctttttaggagactttcatccatctggtcagaaaagacaatttttttattgtcaagcttggtggcaaaggagccatagagggcgttggatagaagcttggcgatggatctcatggtctgatttttgtcacggtcggcgcgctccttggccgcgatgtttagctggacatactcgcgcgcgacgcacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcatccaaggccatctgccactcgcgggcggccagcgctcgctcgtaggggttgaggggcggaccccagggcatgggatgcgtgagggcggaggcgtacatgccgcagatgtcatacacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagtttgaggagatggtgggccgttggaagatgttaaagtgggcgtgaggcaggcggaccgagtcgcggatgaagtgcgcgtaggagtcttgcagcttggcgacgagctcggcggtgacgaggacgtccatggcacagtagtccagcgtttcgcggatgatgtcataacccgcctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtctctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgagcacgatctcgtcgaagccgttgatgttgtgcccgacgatgtagagttccatgaatcgcgggcggcctttgatgtgcggcagctttttgagctcctcgtaggtgaggtcctcggggcattgcagtccgtgctgctcgagcgcccactcctggagatgtgggttggcttgcatgaaggaagcccagagctcgcgggccatgagggtctggagctcgtcgcgaaagagacggaactgctggcccacggccatcttttcgggtgtgacgcagtagaaggtgagggggtcccgctcccagcgatcccagcgtaagcgcacggcgagatcgcgagcgagggcgaccagctcggggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaagcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctccacggtggcggcgaggtcattcgagatgcgacccattagctgcgagaaggcgcccaggccgctctcgttccagacgcggctgtagaccacgtccccgtcggcgtcgcgcgcgcgcatgaccacctgcgcgaggttgagctccacgtgccgcgtgaagacggcgtagttgcgcaggcgctggaagaggtagttgagggtggtggcgatgtgctcggtgacgaagaagtacatgatccagcggcgcaggggcatctcgctgatgtcgccgatggcctccagcctttccatggcctcgtagaaatccacggcgaagttgaaaaactgggcgttgcgggccgagaccgtgagctcgtcttccaggagccggatgagctcggcgatggtggcgcgcacctcgcgctcgaaatccccggggacctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttaaggcggcggatggtggcgaggaggaccaggtccttgggtcccgcttgctggatgcggagccgctcggccatgccccaggcctggccctgacaccggctcaggttcttgtagtagtcatgcatgagcctttcaatgtcatcactggcggaggcggagtcttccatgcgggtgaccccgacgcccctgagcggctgcacgagcgccaggtcggcgacgacgcgctcggcgaggatggcctgttgcacgcgggtgagggtgtcctggaagtcatccatgtcgacgaagcggtggtaggccccggtgttgatggtgtaggtgcagttggccatgagcgaccagttgacggtctgcaggccgggttgcacgacctcggagtacctgagccgcgagaaggcgcgcgagtcgaagacgtagtcgttgcaggtgcgcacgaggtactggtanccgactangaagtgcggcggcggctggcggtagancggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaataatccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgccgaccgagtggggtcgctagacttgaaagcggccgaaaaccccgccgggtagtggctcgcgcccgtagtctggagaagcatcgccagggttgagtcgcggcagaacccggttcgcggacggccgcggcgagcgggacttggtcaccccgccgatttaaagacccacagccagccgacttctccagttacgggagcgagcccccttttttctttttgccagatgcatcccgtcctgcgccaaatgcgtcccacccccccggcgaccaccgcgaccgcggccgtaacaggcgccggcgctagccagccacagacagagatggacttggaagagggcgaagggctggcgagactgggggcgccgtccccggagcgacacccccgcgtgcagctgcagaaggacgtgcgcccggcgtacgtgcctgcgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatagcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgatcaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtatcgcaacgaccgcatccacaaggccgtgagcacgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaggccgcctacggtccagaggacttggatgaggatgaggaagaggaggaggatgcacccgttgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggaccggctggtgacggacgtgcgcgaggccgtggcgcagcgcgagcggttcaagaacgagggcctgggctcgctggtggcgctgaacgccttcctggcgacgcagccggcgaacgtgccgcgagggcaggacgattacaccaactttatcagcgcgctgcggctgatggtgaccgaggttccccagagcgaggtgtaccagtcgggcccggactactttttccagacaagccggcagggcctgcagacggtgaacctgagtcaggctttcaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcgcaggtggacgagcagaccttccaggagatcacgagcgtgagccgcgcgctggggcagaacgacaccgacagtctgagggccaccctgaactttttgctgacaaatagacagcagaagatcccggcgcagtacgcactgtcggccgaggaggaaaggatcctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacctgcaccgcgcggcggccatgaacacggactactttacaaacgccatattgaacccgcactggcttccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgagtacctgaacgactcgctgctgcagccgccgcgggtcaagaacgccatggccaataacgggatagagagtctggtggacaaactgaaccgctggaagacctacgctcaggaccatagggagcctgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccagactggggcggcggatgttttgaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcatcccaaagatgtagatgccagtgatttaagcaaggatatcttagagtataagtggtttgagtttaccctgcccgagggcaacttttccgagaccatgaccatagacctgatgaacaacgccatcttggaaaactacttgcaagtggggcggcagaatggcgtgctggagagcgatatcggagtcaagtttgacagcaggaatttcagactgggctgggacccggtgaccaagctggtgatgccaggggtctacacctacgaggccttccacccggacgtggtgctactgccgggctgcggggtggacttcaccgagagccgcctgagcaacctcctgggcattcgcaagaagcaaccttttcaagagggcttcagaatcatgtatgaggatctagaagggggtaacatccccgctctcctggataccaaaaaatatctggatagcaagaaggaacttgaggatgctgccaaggaagctgcaaagcaacagggagatggtgctgtcactagaggcgatacccacctcactgtagctcaagaaaaagcagctgaaaaggagctagtgatcgtaccaattgaaaaggatgagagcaacagaagttacaacctgatcaaggacacccatgacaccctgtaccgaagctggtacctgtcctatacctacggggaccccgagaagggggtgcagtcgtggacgctgctcaccaccccggacgtcacctgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgcttccgctgcgcagcagtatccgcggagtccagcgagtgaccgtcactgacgcccgtcgccgcacctgtccctacgtctacaaggccctgggcatagtcgcgccgcgcgtgctctccagtcgcaccttctaaaaaatgtctattctcatctcgcccagcaataacaccggctggggtcttactaggcccagcaccatgtacggaggagccaagaagcgctcccagcagcaccccgtccgcgtccgcggtcacttccgcgctccctggggagcttacaagcgggggcgcactgccaccgccgccgccgtgcgcaccaccgtcgacgacgtcatcgactcggtggtcgccgacgcgcgcaactacacccccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgccgccactgcaccccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggacccccggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccaccgctaccgtctccacttttaccgccgccacggctaccgagcctcccaggaggcgaagatggggcgccgccagccggctgatgcccaactacgtgttgcatccttccatcatcccgacgccgggctaccgcggcacccggtactacgccagccgcaggcgcccagccgccaaacgccgccgccgcantgccacccgccgccgtntggcccccgcccgcgtgcgccgcgtaaccacgcgccggggccgctcgntcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcgggtttctgcccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcggccgcccacggtggtggagatggaagatgcaactcttccgccgcccaaaggcgagaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccacgcccgctccaccgaaggcagctccggttgtgcaggcccctccggtggcgaccgccgtgcgccgcgtccccgcccgccgccaggcccagaactggcagagcacgctgcacagtatcgtgggcctgggagtgaaaagtctgaagcgccgccgatgctattgagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccatgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagtttggcccccaagggcgcccccaactccagtcagtggactgacaaagaacggcaaaatggtggacaaccacccactacaaaagatgttacaaaaacattcggagtagcagccaggggagggcttcatattactgataaaggactacaaataggagaagatgaaaataacgaggatggtgaagaagagatatatgcagacaaaactttccagccagaacctcaagtaggagaggaaaactggcaagatactgatgttttctatggcggcagagcgcttaaaaaggaaaccaaaatgaaaccatgctatggctcttttgccagacctaccaatgaaaaaggaggtcaagctaaatttttaaatggcgaaaacggtcaaccttctaaagatcaagatattacattagctttctttgatcttaaacaaaatgacactggaactactcaaaaccagccagatgttgtcatgtacactgaaaatgtgtatctggaaaccccagacacccatgtggtgtacaaacctggcaaggaagatacaagctccgctgctaaccttacacaacagtccatgcccaacaggcccaactacattggtttcagggacaactttgtggggctcatgtattacaacagcactggcaacatgggtgtgctggctggtcaggcctctcagttgaatgctgtggttgacttgcaagacagaaacaccgagctgtcttatcagctcttgctagattctctgggtgacagaaccagatactttagcatgtggaattctgcggtggacagctatgatccagatgtcaggatcattgagaatcacggtgttgaagatgagcttccaaattattgcttcccactggatggatctggcagcaataccgcatatcaaggtgttaaatatgaaaacggagctggcaatggaagctggaaagtagatggcgaagttgcttctcagaatcagatcgccaagggtaatctgtatgccatggagataaaccttcaggccaacctgtggaagagttttctgtactcgaacgtggcgctgtatctaccagactcctacaagtacacgccggccaacatcacgctgcccaccaacaccaacacctacgagtacatgaacggccgcgtggtggcaccctcgctggtggatgcctatgtcaacatcggtgcccgctggtcgctggaccccatggacaacgtcaaccccttcaaccaccaccgcaacgcgggtctgcgctaccgctccatgcttctgggcaacggccgctacgtgcccttccacatccaagtgccccaaaagttctttgccatcaagaacctgctcctgcttcccggttcctacacctacgagtggaacttccgcaaggatgtcaacatgatcctgcaaagttccctcggcaacgacctgcgcgtcgacggcgcctccgtccgcttcgacagcgtcaacctctatgccaccttcttccccatggcgcacaacaccgcctccacccttgaagccatgctgcgcaacgacaccaacgaccagtccttcaacgactacctctcggccgccaacatgctctacccaatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccattccctacctcgacggaaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgactaaggactggttcctcgtccagatgctctctcattacaacattggctaccagggcttctacgtgcctgacggttacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccataccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggaaacgccaccacataagcatgagcggctccagcgaacgagagctcgcggccatcgtgcgcgacctgggctgcgggccctactttttgggaacccacgacaagcgcttccctggcttcctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttctgttgcatgttcttgcatgccttcgtgcactggcccgaccgacccatggacgggaaccccaccatgaacttgctgacgggggtgcccaacggcatgctacaatcgccacaggtgctgcccaccctccggcgcaaccaggaggagctctaccgcttcctcgcgcgccactccccttactttcgatcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggaagccacttgaactcggggatcaccagtttgggaaccggaatctcggggaaggtctcgctccacatgcgccggctcatctgcagggcgcccagcatgtccggggcggagatcttgaaatcgcagttgggaccggtgctctgcgcgcgcgagttgcggtacacggggttgcagcactggaacaccatcagactggggtacttcacactggccagcacgctcttgtcggtgatctgatccttgtcaaggtcctcggcgttgctcaggccaaacggggtcatcttgcacagctggcggcccaggaagggcacgctctgaggcttgtggttacactcgcagtgaatgggcattagcatcatccccgcgccgcgctgcatattcgggtagagggccttgacaaaggccgagatctgtttgaaagcttgctgggccttggctccctcgctgaaaaacagcccgcagctcttcccgctgaactggttattcccgcaaccggcatcctgcacgcagcagcgcgcgtcatggctggtcagttgcaccacgctccgtccccagcggttctgggtcaccttggccttgctgggttgctccttcagcgcgcgctgtccgttctcgctggtcacatccatctccaccacgtggtctttgtggatcatcaccgttccatgcagacacttgagctggccttccacctcggtgcagccgtgatcccacagggcgcatccggtgcactcccagttcttatgcgcgatcccgctgtggctgaagatgtaaccttgcaacaggcgacccatgacggtgctaaaggctttctgggtggtgaaggtcagttgcagaccgcgggcctcctcgttcatccaggtctggcacatcttctggaagatctcggtctgctctggcatgagcttgtaagcatcgcgcaggccgctgtcgacgcggtagcgttccatcagcacgttcatggtatccatgcccttctcccaggacgagaccagaggcagactcagggggttgcgcacgttcaggacaccgggggtcgcgggctcgacgatgcgttttccgtccttgccttccttcaacagaaccggaggctggctgaatcccactcccacgatcacggcgtcttcctggggcatctcttcgtctgggtctacctttgtcacatgcttggtctttctggcttgcttcttttttggagggctgtccacggggaccacgtcctcctccgaagacccggagcccacccgctgatactttcggcgcttggtgggcagaggaggtggcggcgaggggctcctctcctgctccggcggatagcgcgccgacccgtggccccggggcggantggcctctcgctccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacctcttctcgccgcgcgtgccccccaaacgccagcccaacggcacctgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggtcgtgctcaagcgcagcgtcgagctcacccacttcgcctaccccgccgttaacctcccgcccaaggtcatgcgtcgcatcatggatcagcttatcatgccccacatcgaggccatcgatgagacccaagagcagcgccccgaggacgcccggcccgtggtcagcgacgagatgctcgcgcgctggctcgggacccgcgacccccaggctttggaacagcggcgcaagctgatgctggccgtagtcctggtcaccctcgagctcgaatgcatgcgccgcttcttctgcgaccccgagaccctgcgcaaggtcgaggagaccctgcactacactttcagacacggtttcgtcaggcaagcctgcaagatctccaacgtggagctgaccaacctggtctcctgcctggggatcctgcatgagaaccgcctggggcagacagtgctccactctaccctgaagggcgaggcacggcgggactatgtccgcgactgcgtctttctctttctatgccacacatggcaagcagccatgggcgtgtggcagcagtgtctcgaggacgagaacctgaaggagctggacaagcttcttgctagaaaccttaaaaagttgtggacgggcttcgacgagcgcaccgtcgcctcggacctggccgagatcgttttccccgagcgcctgaggcatacgctgaaaggcgggctgcccgacttcatgagccagagcatgttgcaaaactaccgcactttcattctcgagcgctcgggtatcctgcccgccacctgcaacgccttcccctccgactttgtcccgctgagctaccgcgagtgtcccccgccgctgtggagccactgctacctcttgcagctggctaactacatctcctaccactcggacgtgatcgaggacgtgagcggcgaggggctgctcgagtgccactgccgctgcaacctgtgctccccgcaccgctccctggtctgcaacccccagctcctgagcgagacccaggtcatcggtaccttcgagctgcaaggtccggagaagtccaccgctccgctgaaactcacgccggggttgtggacttccgcgtacctgcgcaaatttgtacccgaagactaccacgcccatgagataaagttcttcgaggaccaatcgcgtccgcagcacgcggatctcacggcctgcgtcatcacccagggcgcgatcctcgcccaattgcatgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccggcggtcacggatacaacctccgctccggtcaagcctcctcgtagatgggatcgagtgaagggtgacggtaagcacaagcggcagggctaccgatcatggagggcccacaaagccgcgatcatcgcctgcttgcaagactgcggggggaacatcgctttcgcccgccgctacctgctcttccaccgcggggtgaacatcccccgcaacgtgttgcattactaccgtcaccttcacagctaagaaaaagcaagtaagaggagtcgccggaggaggcctgaggatcgcggcgaacgagccctcgaccaccagggagctgaggaaccggatcttccccactctttatgccatttttcagcagagtcgaggtcagcagcaagagctcaaagtaaaaaatcggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacgggtcaacggggtccgtaaccatcgaaaccagatattgttggagcaggcggcggtcacatccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctgatgatccgaggcagaggcacacagctcaacgacgagttggtgagctcttcgatcggtctgcgaccggacggagtgttccaactagccggagccgggagatcatccttcactcccaaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagctgcccgaggagcaccccaacggccctgcacacggagtacggatcaccgtagagggcaccgccgagtctcacctggtcaggttcttcacccagcaacccttcctggtcgagcgggaccggggcgccaccacctacaccgtctactgcatctgtcctaccccaaagttgcatgagaatttttgctgtactctttgtggtgagtttaataaaagctgaactaagaacctactttggaatcccttgtcgtcatcaaatccacaagaccatcaacttcacctttgaggaacaggtgaactttacctgcaagccacacaagaagtacgtcacctggttttaccagaacactactctagcagtagccaacacctgctcgaacgacggtgttcttcttccaaacaatctcaccagtggactaactttctcagtgaaaagggcaaagctaattcttcatcgccctattgtagaaggaacttaccagtgtcagagcggaccttgcttccacagtttcactttggtgaacgttaccggcagcagcacagtcgctccagaaactaaccttctttctgatactaacactcctaaaaccggaggtgagctctgggttccctctctgacagaggggggtagtcatattgaagcggtcgggtatttgattttaggggtggtcctgggtgggtgcatagcggtgctatattaccttccttgctgggtcgaaatcagggtatttatctgctgggtcagacattgtggggaggaaccatgaaggggctcttgctgattatcctttccctggttgggggtttactggcctgccacgaacagccacgatgtaacatcaccacaggcaatgagaggaacgactgctctgtagtgatcaaatgcgagcaccagtgtcctctcaacattacattcaagaataagaccatgggaaatgtatgggtgggattctggcaaccaggagatgagcagaactacacggtcactatccatggtagcgatggaaatcacactttcggtttcaaattcatttttgaagtcatgtgtgatatcacactgcatgtggctagacttcatggcttgtggccccctaccaaggagaacatggttgggttttctttggcttttgtgatcatggcctgtgcaatgtcaggtctgctggtaggggctctagtgtggttcctgaagcgcaagcccaggtacggaaatgaggagaaggaaaaattgctataaatctttttctcttcgcagaaccatgaatactttgaccagtgtcgtgctgctctctcttttagttattaatgtggaatgtgccgatcctattctagttagtgtagattggggaaaaaatcttacattagagggtcctaaagaaacaccagttgaatggtggggtggaagaaacatacaacaactgtgcatagggaatcaaaccaaacataaagagctaagtcacagatgtaatgtccagaacataactttactgtttgtaaatactagttttaatggagactactttgggtttaaaaatgataacagcggtatgaaacattataaagtcacagttataccccctaaaccctccactcggaaacctctttctcctccacactatgtaaacgcaactatggggcaaaacctaacattagtggggcctgcaaacattccagttacttggcttagtgaatatggcacgttgtgtgagggcaaaaaaattttgcacaaagnaattaaatcacacctgtaacgaacagaacctcacgttactgtttgttaatatgacacacaacggaccatattttggctttgacaaatacaacattgatagagagcagtatgaggtttctattattagtttgtttaaagttggcgctggacagaagaaaattgggaaaggacagaaaaaggaggaaaagacaaaaccaaactctagtgatttgggacaaagacaatccagaccaaagaaaaaagatattgttgaagaggtccaaatcaaaacaggagaaaatcgaacccttgttggtccacctggaaaagttgattggattaaactttccagtggaaacaataatgttcttaagttgtgtaatggcgacaagtatattaaacacacatgtgatggtcaaaatttaacattaattaatgtgactagaatttatgacggaacttattatggttctagcaatgatggctcaagtcattacaaagttaccatctatgaattacacaaagttaataaaactaaatctatgcttaagccatacactacaaaaagaactacagtgaatgcaacagatgacagtgctcacaaaattgctttgcagcaggaaaataatgggcaaacagaaaatgatcaagaatcaaaaattccatctgctactgtggcaatcgtggtgggagtgattgcgggcttcataactataatcattgtcattctgtgctacatctgctgccgcaagcgtcccagggcatacaataatatggtagacccactactcagcttctcttactgagactcagtcactttcatttcagaaccatgaaggctttcacagcttgcgttctgattagcataattacacttagtttagcagcacctaaaccagaagtatatacacaagttaatgtcactaggggtgggaatgctacactagatggaccatttaacaataacacatggacaagatatcatgatgatgggagaaaaaacggatggatgaatatttgtaaatggtcagacccatcatacacatgtcatagtaatggaagccttagtatttttgctttcaacattagttcaggtaaatataaagttcaaagttacactaacagttataatggattagatggttatgaaaaacttgaagttaaaatgtttaatctaacagtaattgagcctccaaccactagagcacccaccacagttaggacaactaaggaaacaacacagcctaccactgtacccactacacatccaaccaccacagtcagtacaactattgagaccactactcatactacacagctagacacaacagtgcagaatactactttactgattgaatttttactaagagggaatgaaagtactactgatcagacagaggctacctcaagtgccttcagcagtactgcaaatttaacttcgcttgcttggactaatgaaaccggagtatcattgatgcatggccagccttactcaggtttggatattcaaattacttttctggttgtctgtgggatctttattcttgtggttcttctgtactttgtctgctgcaaagccagagagaaatctagtaggcccatctacaggccagtaatcggggaacctcagcctctccaagtggaagggggtctaaggaatcttctcttctctttttcagtatggtgatcagccatgattcctaggttcttcctatttaacatcctcttctgtctcttcaacatctgcgctgccttcgcggccgtctcgcacgcctcgcccgactgtctcgggcccttccccacctacctcctctttgccctgctcacctgcacctgcgtctgcagcattgtctgcctggtcgtcaccttcctgcagctcatcgactggtgctgcgcgcgttacaattatctccaccacagtcccgaatacagggacaagaacgtagccagaatcttaaggctcatctgaccatgcagactctgctgatactgctatccctcctctcccctgcccttgctgactgtaaatttgcggacatatggaatttcttagactgttatcaagagaaaatggatatgccttcctattacttggtgattgtgggtgtagtcatggtctgctcctgcactttctttgctatcatgatctacccctgttttgatctcggctggaactctgttgaggcattcacatacacactagaaagcagttcactagcctccacgccgccacccacaccgcctccccgcagaaatcagttccccctgattcagtacttagaagagccccctccccggcccccttccactgttagctactttcacataaccggcggcgatgactgaccaccacctggacctcgagatggacggccaggcctccgagcagcgcatcctgcaactgcgcgtccgtcagcagcaggagcgggccgccaaggagctcctcgatgccatcaacatccaccagtgcaagaagggcatcttctgcctggtcaaacaggcaaagatcacctacgagctcgtgtccaacggcaaacagcatcgcctcacctatgagatgccccagcagaagcagaagttcacctgcatggtgggcgtcaaccccatagtcatcacccagcagtcgggcgagaccagcggctgcatccactgctcctgcgaaagccccgagtgcatctactccctcctcaagaccctttgcggacttcgcgacctcctccccatgaactgattgattaaagcccagaaaccaatcaaacccccttccccatcaccccaaataacaatcattggaaataatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatgtcaaagaggctccgggtggaagatgacttcaaccccgtctacccctatggctacgcgcggaatcagaatatccccttcctcactcccccctttgtctcctccgatggattccaaaacttcccccctggggtcctgtcactcaaactggctgatccaatcgccatcgccaatgggaatgtctcactcaaggtgggagggggactcactgtagaacaacagtctggaaaactgagtgtggatactaaggcacccttgcaagttgcaaatgacaacaaattggagctatcttatgatgatccatttaaggtagagaataacaaacttggaattaaagctggccatggtttagcagttgtaactaaagaaaacacaagtcttcctagtctagttggaacacttgtagttttaactggaaaaggaataggtactggatcaagtgcacatggaggaactattgatgtaagacttggtgaaggaggtgggttatcatttgatgaaaaaggagacttagtagcttgggacaaaaaaaatgatacacgcaccctttggacaacacctgatccttctccaaattgcaaagttgaaacagcaagagactcaaagctaaccttagcacttacaaaatgtggtagtcaaattttggccactgtatctttacttgttgttacgggcaaatatgctattataagtgacacagtcaacccaaagcagttctctattaagttactgtttaatgacaagggtgttttgttaagtgactcaaatcttgatgggacatattggaactatagaagcaacaataacaacataggcactccttataaagaggctgttggttttatgccaagcacaacagcttatcctaagccaaccaacaacaccagcacagatccggataaaaaagtgagtcaaggtaaaaataaaattgtaagcaatatttatcttggaggagaggtatatcaaccaggatttattgttgttaaatttaatcaggaaactgatgccaattgtgcatactctattacatttgattttggatggggtaaggtgtataaggatcctataccatatgatacctcttcttttactttctcatatatcgctcaagaatgaaagaccaataaacgtgtttttcattgaaaattttcatgtatctttattgatttttacaccagcacgggtagtcagtctcccaccaccagcccatttcacagtgtaaacaattctctcagcacgggtggccttaaataggggaatgttctgattagtgcgggaactggatttagtgtctataatccacacagtttcctggcgagccaaacggggatcggtgattgagatgaagccgtcctctgaaaagtcttccaagcgggcctcacagtccaaggtcacagtctggtggaatgagaagaacgcacagattcatactcggaaaacaggatgggtctgtgcctctccatcagcgccctcaacagtctctgccgccggggctcggtgcggctgctgcagatgggatcgggatcgcaagtctctctgactatgatccccacagccttcagcatcagtctcctggtgcgtcgggcacagcacctcatcctgatctcgctcatgttctcacagtaagtgcagcacataatcaccatgttattcagcagcccataattcagggtgctccagccaaagctcatgttggggatgatggaacccacgtgaccatcataccaaatgcggcagtatatcaggtgcctgcccctcatgaacacactgcccatatacatgatctctttgggcatgtttctgttcacaatctggcggtaccaggggaagcgctggttgaacatgcacccgtaaatgactctcctgaaccacacggccagcanggtgcctcccgcccggcactgcagggagcccggggacgaacagtggcaatgcaggatccagcgctcgnacccgctcaccatctgagctctcaccaagtccagggtagcggggcacaggcacactgacatacatctttttaaaatttttatttcctctggggtcaggatcatatcccaggggactggaaactcttggagcagggtaaagccagcagcacatggtaatccacggacagaacttacattatgatattcagcatgatcacaatcgggcagcagggggtgttgttcagtcagtgaggccctggtctcctcctcagatcgtggtaaacgggccctgcggtatggatgatggcggagcgaggtcgattgttcctcggtgctcattgtagtgcaccctcttgcgtaccttgtcgtacttctgccagcagaaagtggcccgggaacagcagatacccctcctccgtccgtcctttcgctgctgccgctcagtcatccaactgaagtacatccattcccgaaggttctggagaagttcctctgcatctgatgaaacaaaaagcccgtccatgcgaattcccctcatcacatcagccaggactctgtaggccatccccatccagttaatgctgccttgtctatcattcagagggggcggtggcaggattggaagaaccattatttttttactccaaacggtcgcgcagcaatataaaattcaggtcacggaggtggcacctctctcctccactgttttggtggaaacagacagccaaatcaaaaattatgcgattctcaaggtgctctactgtggcttccagcagaggctccacacgtacatccagaaacaacagcacattaaaagcgggcccgccatcctgctcntcaatcatcatattacagtcctgaaccatccccaggtaattttcgtttttccagccttgaattatcgctacaaactgttggtgtaagtccactccacacatgttgaaaagctcccacagcgccccctccaccttcataagcaggcagaccttcataatagcaacagatctggctgctccacaacctgcagcgtgttcaaaacaacaagattcaatgagtttctgccctctgccctgagctcgcgtctcagtgttagctgtaaaaagtcactcaagtcctcggccactacagatgccaagtcagagccagggctaagcgttggactggcaagcgtgagggagtatcttagagctccataattagcccccaaaaactgcatgctggaataagctctctttgtgtcaccggtgattccttccaagaggtgagtgataaagcgaggtaggtgctctttaatcatagcagtaatggaaaagtcctctaaataagtcactagggccccagggaccacaatgtggtagctgacaccgcgccgctcaagcatggttagtagagatgagagtctgaaaaacagaaagcatgcactaaaccagggtggctattctcactgaaggaaaaatcactctctccagcagcagggttcccactgggtggccctcgcggacatacaaaaatcggtccgtgtggttaaagagcagcacagagagatcatgtcttctcccggcaaatatcacatcggactgggttagtatgcccctggcatggtagtcattcaaggccataaatctgccctgatatccagtaggaaccagcacgctcactctgaggtgaagcaatatcaccccatgcggaggaatgtggaaagattcggggcaaaagaagatatatctattgctagccccttcctggacgggagcgatccctccagggctatctatgaaagcatacagagattcagccatagctcagcccgcttaccagtagacagagagcacagcagtacaagcgccaacagcagcgactgactacccactgacccagctccctatttaaaggcaccttacactgacgtaatgaccaaaggtctaaaaaccccgccaaaaaaaaacacacacgccctgggtgtttttcgcgaaaacacttccgcgttgtcacttcctcgtatcgatttcgtgactcaacttccgggttcccacgttacgtcacttctgcccttacatgtggctccgccgtagggcgccatcttgcccacgtccaaaatggcttccatgtcagaccacgcctccgcgccgaccgttgacgccgtgcgtcgtgacgtcagtaacggctcttccaacagccaatcagcgtcggccccgccccaaattcaaaagctcatttgcatattaacttttgtttactttgtggggtatatttattgatgatg

(19) United States
(12) Patent Application Publication
(10) Pub. No.: US 2013/0178517 A1
(43) Pub. Date: Jul. 11, 2013

(54) Methods And Compositions For Treatment Of Lipogenic Virus Related Conditions

(71) Applicants: Richard L. Atkinson, Mechanicsville, VA (US); Zendra Pei-lun Lee, Glen Allen, VA (US); Jia He, Richmond, VA (US)

(72) Inventors: Richard L. Atkinson, Mechanicsville, VA (US); Zendra Pei-lun Lee, Glen Allen, VA (US); Jia He, Richmond, VA (US)

(21) Appl. No.: 13/809,209

(22) PCT Filed: Jul. 8, 2011
(86) PCT No.: PCT/US2011/043399

§ 371 Date: Mar. 22, 2013

Related U.S. Application Data
(60) Provisional application No. 61/362,915, filed on Jul. 9, 2010.
(60) Provisional application No. 61/434,984, filed on Jan. 21, 2011.

(51) Int. Cl. A61K 31/353; A61K 31/155; A61K 31/15; A61K 31/166

(52) U.S. Cl. CPC A61K 31/353; A61K 31/166; A61K 31/155; A61K 31/15
USPC 514/456; 514/622; 514/635; 514/633

(57) ABSTRACT

Disclosed are methods of treatment for adipogenic virus-related conditions. The methods may comprise administering a composition comprising an effective dose of an antiviral agent to a subject having an adipogenic adenovirus-related condition. Administration of the antiviral agent may prevent or reduce viral proliferation in the subject. The method may also include administering a composition comprising an effective dose of a therapeutic agent known to treat the adipogenic adenovirus-related condition in conjunction with the composition comprising an effective dose of an antiviral agent. Administration of the antiviral agent and the therapeutic agent may reduce or eliminate one or more symptoms of the adipogenic adenovirus-related condition more efficiently than administration of either the antiviral agent and the therapeutic agent alone.

### CROSS REFERENCE TO RELATED APPLICATIONS

The present application claims priority from U.S. Provisional Patent Application Ser. No. 61/362915, filed Jul. 9, 2010, and U.S. Provisional Patent Application Ser. No. 61/434984, filed Jan. 21, 2011. The disclosure of U.S. Provisional Patent Application Ser. Nos. 61/362915 and 61/434984 are hereby incorporated by reference in their entireties herein.

### FIELD OF THE INVENTION

The invention relates to methods and compositions for treating lipogenic adenovirus-related conditions. More particularly, the invention relates to methods of administering at least one anti-viral agent, alone or in combination with a therapeutic agent for treatment of a lipogenic adenovirus-related condition, to a subject with the lipogenic adenovirus-related condition, wherein the subject is infected with a lipogenic adenovirus. The invention also relates to therapeutic compositions for reducing or eliminating one or more symptoms of the lipogenic adenovirus-related condition. In some aspects, the invention relates to method and compositions for reducing the incidence of or preventing lipogenic adenovirus-related conditions.

### BACKGROUND OF THE INVENTION

Human lipogenic adenoviruses, such as adenovirus-36 (Ad-36), cause obesity in humans and non-human animals, are associated with certain cancers in humans, and also cause or contribute to a number of diseases due to the complications of obesity and/or by altering cell fatty acid biochemistry, and also affect the outcome of regimens that affect body weight. See U.S. Pat. Nos. RE39544; RE39914; RE42129; 7,442,511; 7,507,418; and 7,745, 110, the disclosures of which are incorporated by reference in their entirety.

Lipogenic adenovirus-related diseases may include, inter alia, diabetes mellitus, hypertension, hyperlipoproteinemia, cardiac disease such as atherosclerotic disease and congestive heart failure, pulmonary diseases such as sleep apnea and asthma, cerebrovascular accidents, cancers such as breast, uterus colon and prostate cancer, neurodegenerative disease such as Alzheimer's disease, gall bladder disease such as stones and infection, toxemia during pregnancy, risks during surgery, gout, decreased fertility, degenerative arthritis, and early mortality.

The adenovirus E4 region, specifically the E4orfl gene, has been shown to play a role in lipogenic adenovirus-induced cancers. In particular, the Ad-36 E4orfl gene has been shown to be involved in producing obesity by a direct effect on adipocyte metabolism, and the E4orfl region of human adenovirus-5 has been shown to be an oncogene that produces obesity in mice. These findings show a direct link between obesity and cancer, with both being due to a human adenovirus, and provide the likely mechanism via a viral gene.

Ad-36 has been shown to increase glucose transport into cells and increase AKT and PI3-kinase enzyme activity. See Schafer, et al., Nature 461, 109-113 (2009) and Wang, et al., Diabetes, 57:1805-13 (2008). The AKT and PI3-kinase pathways play a role in glucose transport and apoptosis, both of which are important in cancer growth and cancer metastasis, as well as a number of various diseases and conditions.

### SUMMARY OF THE INVENTION

The invention provides methods and compositions for treating lipogenic adenovirus-related conditions. The invention may be embodied in a variety of ways.

In some embodiments, the invention provides a method for treating a lipogenic adenovirus-related condition comprising: a) identifying a subject having a lipogenic adenovirus-related condition, wherein the subject is infected with a lipogenic adenovirus; and b) administering a composition comprising an effective dose of an antiviral agent to the subject, wherein administration of the antiviral agent prevents or reduces lipogenic adenovirus proliferation and/or action.

In some aspects, the invention provides a method that also has a step of administering a composition that includes an effective dose of a therapeutic agent known to treat the lipogenic adenovirus-related condition in conjunction with the composition comprising an effective dose of an antiviral agent, wherein administration of the antiviral agent and the therapeutic agent reduce or eliminate one or more symptoms of the lipogenic adenovirus-related condition more efficiently than administration of either the antiviral agent or the therapeutic agent alone.

In some aspects of the invention, the step of identifying a subject having a lipogenic adenovirus-related condition involves determining whether a nucleic acid sequence specific to the lipogenic adenovirus is present in the biological sample. In addition, in some aspects of the invention, the step of identifying a subject having a lipogenic adenovirus-related condition involves determining whether antibodies specific to the lipogenic adenovirus are present in the biological sample. Also, in some aspects of the invention, the step of identifying a subject having a lipogenic adenovirus-related condition includes determining whether lipogenic adenovirus proteins and/or lipogenic adenovirus particles are present in the biological sample.

In some aspects, the invention provides methods for treating a lipogenic adenovirus-related condition. In some aspects, the lipogenic adenovirus-related condition is selected from the group consisting of cancer, obesity, diabetes, pancreatic dysfunction, liver disease, liver dysfunction, cirrhosis, muscle dysfunction, pulmonary dysfunction, brain and nervous system dysfunction, and adrenal dysfunction. In some aspects of the invention, the lipogenic adenovirus-related condition comprises cancer. In some aspects of the invention, the cancer is one or more of prostate cancer, breast cancer, uterine cancer, ovarian cancer, colon cancer, lung cancer, kidney cancer, and pancreatic cancer. In some aspects of the invention, the lipogenic adenovirus-related condition comprises diabetes mellitus. In some aspects of the invention, the lipogenic adenovirus-related condition comprises Alzheimer's disease.

In some aspects of the invention, the subject treated by the method is a human. In some aspects of the invention, the subject is mammalian or avian.

In some aspects of the invention, the lipogenic adenovirus comprises one or more of adenovirus type 5, adenovirus type 36, and adenovirus type 37. In some aspects of the invention, the lipogenic adenovirus comprises adenovirus type 36.

In some aspects of the invention, the antiviral agent comprises one or more of a ribonucleotide reductase inhibitor, a nucleoside analog, a nucleotide analog, a protease inhibitor, an antisense drug, a ribozyme, a trace mineral binder, an antioxidant, an AMP-activated protein kinase (AMPK) activator, and/or an interferon drug.

In some aspects of the invention, the antiviral agent comprises one or more of Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lamivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, Zidovudine, conjugated Linoleic acid, Echinacea, Elder berry, Garlic, Hyssop, Kahalalide F, Licorice Root, Lycoris radiate, St. John's Wort, Uncaria tomentoas, Zostrix, metformin, luteolin, conjugated linoleic acid, N-acetylcysteine, monolaurin, alpha lipoic acid, melatonin, and any combination thereof.

In some aspects of the invention, the antiviral agent is administered intranasally, orally, or by injection intravenously, intramuscularly, subcutaneously, and/or peritoneally.

In some aspects of the invention, he therapeutic agent is a chemotherapeutic agent.

In some aspects of the invention, the therapeutic agent comprises one or more of an alkylating agent, an antimetabolite, an anthracycline, a plant alkaloid, a topoisomerase inhibitor, a cytotoxic antibiotic, a targeted therapeutic or a hormone.

In some aspects of the invention, the therapeutic agent comprises one or more of cisplatin, carboplatin, oxaliplatin, mechlorethamine, cyclophosphamide, chlorambucin, ifosfamide, azathioprine, mercaptopurine, thioguanine, fludarabine, pentostatin, gemcitabine, cladribine, vincristine, vinblastine, vinorelbine, vindesine, podophyllotoxin, etoposide, teniposide, paclitaxel, docetaxel, irinotecan, topotecan, amsacrine, etoposide, etoposide phosphate, teniposide, actinomycin, aclarubicin, bleomycin, dactinomycin, daunorubicin, doxorubicin, epirubicin, idarubicin, mitomycin, valrubicin, plicomycin, mitozantrone, tamoxifen, anastrozole, letrozole, fulvestrant, capecitabine, trastuzumab or metformin.

In some aspects of the invention, the step of administering the composition that includes the antiviral agent inhibits and/or reduces expression of lipogenic enzymes and/or lipogenic transcription factors. In some aspects of the invention, the lipogenic enzymes comprise one or more of fatty acid synthase (FAS), glycerol-3-phosphophate dehydrogenase (GPDH), lipoprotein lipase (LPL), stearoyl-CoA desaturase 1 (SCD1), carnitine palmitoyltransferase 1 (CPT1), L-type pyruvate kinase (L-PK), proteins in the phosphatidylinositol 3-kinase (P13K) signaling pathway, and proteins in the AKT/Protein Kinase B (PKB) signaling pathway. In some aspects of the invention, the lipogenic transcription factors comprise one or more of peroxisome proliferator-activated receptor gamma (PPAR-γ), CCAAT/enhancer binding protein alpha (C/EBP-α), C/EBP-β, sterol regulatory element-binding protein 1 (SREBP-1), and carbohydrate responsive element- binding protein (ChREBP).

In some aspects of the invention, the step of administering the composition including the antiviral agent inhibits and/or reduces the expression of adipocyte differentiation factors. In some aspects of the invention, the adipocyte differentiation factors comprise one or more of peroxisome proliferator-activated receptor gamma (PPAR-γ), CCAAT/enhancer binding protein alpha (C/EBP-α), C/EBP-β, sterol regulatory element-binding protein 1 (SREBP-1), and carbohydrate responsive element- binding protein (ChREBP).

In some aspects of the invention, the step of administering the antiviral agent reduces or eliminates one or more symptoms of the lipogenic adenovirus-related condition.

In some aspects of the invention, the step of administering the therapeutic agent reduces or eliminates one or more symptoms of the lipogenic adenovirus-related condition.

In some aspects of the invention, the prevention or reduction of lipogenic adenovirus proliferation and/or action reduces cancer aggressiveness.

In some embodiments, the invention provides a therapeutic composition for use in reducing or eliminating one or more symptoms of a lipogenic adenovirus-related condition that includes a) an effective dose of an antiviral agent, wherein administration of the antiviral agent prevents or reduces lipogenic adenovirus proliferation and/or action; b) an effective dose of a therapeutic agent, wherein the therapeutic agent known to treat the lipogenic adenovirus-related condition; and c) a pharmaceutically acceptable carrier.

In some aspects of the invention, the therapeutic composition reduces or eliminates one or more symptoms of the lipogenic adenovirus-related condition more efficiently than administration of either the antiviral agent or the therapeutic agent alone.

In some aspects of the invention, the therapeutic composition includes a antiviral agent that is one or more of a ribonucleotide reductase inhibitor, a nucleoside analog, a nucleotide analog, a protease inhibitor, an antisense drug, a ribozyme, a trace mineral binder, an antioxidant, an AMP-activated protein kinase (AMPK) activator, and/or an interferon drug.

In some aspects of the invention, the therapeutic composition includes an antiviral agent that is one or more of Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lamivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, Zidovudine, conjugated Linoleic acid, Echinacea, Hyssop, Kahalalide F, Licorice Root, Lycoris radiate, St. John's Wort, Uncaria tomentoas, Zostrix, metformin, luteolin, conjugated linoleic acid, N-acetylcysteine, monolaurin, alpha lipoic acid, melatonin, and any combination thereof.

In some aspects of the invention, the therapeutic composition includes a therapeutic agent that is a chemotherapeutic agent. In some aspects of the invention, the therapeutic agent comprises one or more of an alkylating agent, an antimetabolite, an anthracycline, a plant alkaloid, a topoisomerase inhibitor, a cytotoxic antibiotic, a targeted therapeutic or a hormone.

In some aspects of the invention, the therapeutic composition includes a therapeutic agent that is one or more of cisplatin, carboplatin, oxaliplatin, mechlorethamine, cyclophosphamide, chlorambucin, ifosfamide, azathioprine, mercaptopurine, thioguanine, fludarabine, pentostatin, gemcitabine, cladribine, vincristine, vinblastine, vinorelbine, vindesine, podophyllotoxin, etoposide, teniposide, paclitaxel, docetaxel, irinotecan, topotecan, amsacrine, etoposide, etoposide phosphate, teniposide, actinomycin, aclarubicin, bleomycin, dactinomycin, daunorubicin, doxorubicin, epirubicin, idarubicin, mitomycin, valrubicin, plicomycin, mitozantrone, tamoxifen, anastrozole, letrozole, fulvestrant, capecitabine, trastuzumab or metformin.

In some aspects of the invention, the therapeutic composition includes the antiviral agent and/or the therapeutic agent conjugated to an antibody or fragment thereof, wherein the antibody or fragment thereof specifically binds to an extracellular membrane target on cells effected by the lipogenic adenovirus-related condition.

In some aspects of the invention, the therapeutic composition is for use in reducing or eliminating one or more symptoms of a lipogenic adenovirus-related condition, wherein the lipogenic adenovirus-related condition includes cancer, obesity, diabetes, pancreatic dysfunction, liver disease, liver dysfunction, cirrhosis, muscle dysfunction, pulmonary dysfunction, brain and nervous system dysfunction or adrenal dysfunction.

Some embodiments of the invention provide uses of an antiviral agent in preparation of a medicament for treatment of a lipogenic adenovirus-related condition, characterized in that the antiviral agent prevents or reduces lipogenic adenovirus proliferation and/or action.

In some aspects of the invention, the medicament further comprises a therapeutic agent, wherein the therapeutic agent is known to treat the lipogenic adenovirus-related condition.

In some aspects of the invention, the medicament reduces or eliminates one or more symptoms of the lipogenic adenovirus-related condition more efficiently than administration of either the antiviral agent or the therapeutic agent alone.

In some aspects of the invention, the antiviral agent of the medicament includes one or more of a ribonucleotide reductase inhibitor, a nucleoside analog, a nucleotide analog, a protease inhibitor, an antisense drug, a ribozyme, a trace mineral binder, an antioxidant, an AMP-activated protein kinase (AMPK) activator, and/or an interferon drug. In some aspects of the invention, the antiviral agent comprises one or more of Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lamivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, Zidovudine, conjugated Linoleic acid, Echinacea, Hyssop, Kahalalide F, Licorice Root, Lycoris radiate, St. John's Wort, Uncaria tomentoas, Zostrix, metformin, luteolin, conjugated linoleic acid, N-acetylcysteine, monolaurin, alpha lipoic acid, melatonin, and any combination thereof.

In some aspects of the invention, the therapeutic agent of the medicament includes a chemotherapeutic agent. In some aspects of the invention, the therapeutic agent includes one or more of an alkylating agent, an antimetabolite, an anthracycline, a plant alkaloid, a topoisomerase inhibitor, a cytotoxic antibiotic, a targeted therapeutic or a hormone. In some aspects of the invention, the therapeutic agent of the medicament includes one or more of cisplatin, carboplatin, oxaliplatin, mechlorethamine, cyclophosphamide, chlorambucin, ifosfamide, azathioprine, mercaptopurine, thioguanine, fludarabine, pentostatin, gemcitabine, cladribine, vincristine, vinblastine, vinorelbine, vindesine, podophyllotoxin, etoposide, teniposide, paclitaxel, docetaxel, irinotecan, topotecan, amsacrine, etoposide, etoposide phosphate, teniposide, actinomycin, aclarubicin, bleomycin, dactinomycin, daunorubicin, doxorubicin, epirubicin, idarubicin, mitomycin, valrubicin, plicomycin, mitozantrone, tamoxifen, anastrozole, letrozole, fulvestrant, capecitabine, trastuzumab or metformin.

In some aspects of the invention, the antiviral agent of the medicament is conjugated to an antibody or fragment thereof, wherein the antibody or fragment thereof specifically binds to an extracellular membrane target on cells effected by the lipogenic adenovirus-related condition.

In some aspects of the invention, the antiviral agent and/or the therapeutic agent of the medicament are conjugated to an antibody or fragment thereof, wherein the antibody or fragment thereof specifically binds to an extracellular membrane target on cells effected by the lipogenic adenovirus-related condition.

In some aspects of the invention, the medicament for treatment of a lipogenic adenovirus-related condition that include obesity, diabetes, pancreatic dysfunction, liver disease, liver dysfunction, cirrhosis, muscle dysfunction, pulmonary dysfunction, brain and nervous system dysfunction or adrenal dysfunction.

Some embodiments of the invention provide methods of making a therapeutic composition for treatment of a subject having a lipogenic adenovirus-related condition comprising a) selecting an antiviral agent, wherein the antiviral agent prevents or reduces lipogenic adenovirus proliferation and/or action; and b) selecting a pharmaceutically acceptable carrier.

In some aspects of the invention, the method of making a therapeutic composition further includes the step of selecting a therapeutic agent, wherein the therapeutic agent is known to treat the lipogenic adenovirus-related condition.

In some aspects of the invention, the therapeutic composition made by the method reduces or eliminates one or more symptoms of the lipogenic adenovirus-related condition more efficiently than either the antiviral agent or the therapeutic agent alone.

In some aspects of the invention, the antiviral agent in the therapeutic composition made by the method includes one or more of a ribonucleotide reductase inhibitor, a nucleoside analog, a nucleotide analog, a protease inhibitor, an antisense drug, a ribozyme, a trace mineral binder, an antioxidant, an AMP-activated protein kinase (AMPK) activator, and/or an interferon drug. In some aspects of the invention, the antiviral agent in the therapeutic composition made by the method includes one or more of Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lam ivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, Zidovudine, conjugated Linoleic acid, Echinacea, Hyssop, Kahalalide F, Licorice Root, Lycoris radiate, St. John's Wort, Uncaria tomentoas, Zostrix, metformin, luteolin, conjugated linoleic acid, N-acetylcysteine, monolaurin, alpha lipoic acid, melatonin, and any combination thereof.

In some aspects of the invention, the antiviral agent in the therapeutic composition made by the method includes a chemotherapeutic agent. In some aspects of the invention, in the therapeutic composition made by the method includes one or more of an alkylating agent, an antimetabolite, an anthracycline, a plant alkaloid, a topoisomerase inhibitor, a cytotoxic antibiotic, a targeted therapeutic or a hormone. In some aspects of the invention, the therapeutic composition made by the method includes one or more of cisplatin, carboplatin, oxaliplatin, mechlorethamine, cyclophosphamide, chlorambucin, ifosfamide, azathioprine, mercaptopurine, thioguanine, fludarabine, pentostatin, gemcitabine, cladribine, vincristine, vinblastine, vinorelbine, vindesine, podophyllotoxin, etoposide, teniposide, paclitaxel, docetaxel, irinotecan, topotecan, amsacrine, etoposide, etoposide phosphate, teniposide, actinomycin, aclarubicin, bleomycin, dactinomycin, daunorubicin, doxorubicin, epirubicin, idarubicin, mitomycin, valrubicin, plicomycin, mitozantrone, tamoxifen, anastrozole, letrozole, fulvestrant, capecitabine, trastuzumab or metformin.

In some aspects of the invention, the antiviral agent in the therapeutic composition made by the method is conjugated to an antibody or fragment thereof, wherein the antibody or fragment thereof specifically binds to an extracellular membrane target on cells effected by the lipogenic adenovirus-related condition.

In some aspects of the invention, the antiviral agent and/or the therapeutic agent in the therapeutic composition made by the method are conjugated to an antibody or fragment thereof, wherein the antibody or fragment thereof specifically binds to an extracellular membrane target on cells effected by the lipogenic adenovirus-related condition.

In some aspects of the invention, the lipogenic adenovirus-related condition treated by the therapeutic composition made by the method includes cancer, obesity, diabetes, pancreatic dysfunction, liver disease, liver dysfunction, cirrhosis, muscle dysfunction, pulmonary dysfunction, brain and nervous system dysfunction or adrenal dysfunction.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention, are incorporated in and constitute a part of this specification, illustrate aspects of the invention and together with the detailed description serve to explain the principles of the invention. No attempt is made to show structural details of the invention in more detail than may be necessary for a fundamental understanding of the invention and various ways in which it may be practiced.

FIG. 1 illustrates aspects of the invention showing Ad-36 replicates in MCF-10A (Panel A) and MCF-7 (Panel B) cells. Cells were infected with Ad-36 at a MOI of 0.5. At indicated days after infection, cellular DNA was extracted and viral DNA was measured by q-PCR using Ad-36 E4orf-1 Taqman probe and primer set (Applied Biosystem). The data was presented as relative fold change with corresponding Ct value of day 1 post infection.

FIG. 2 illustrates aspects of the invention showing Ad-36 infection promotes cell growth in MCF-10A and MCF-7 cells. Mock- or Ad-36 (MOI 3.8)-infected MCF-10A (Panel A) and MCF-7 (Panel B) cells were plated in 6-well plates at 1×10⁴/well density with complete medium and cultured for the indicated number of days. The cells were harvested from plates with trypsin-EDTA and the numbers of cells were determined by hemocytometer. Quantitative data are the mean±S.D. of triplicates. Concordant results were obtained from multiple independent experiments. *p<0.05 (Student's t test).

FIG. 3 illustrates aspects of the invention showing Ad-36 enhances cell migratory response to FBS. Chemotaxis of mock or Ad-36 MOI 3.8 infected MCF-10A (Panel A) or MCF-7 (Panel B) cells were assessed by transwell assay with 10% FBS as chemoattractant. The cells were loaded to the upper wells and allowed to migrate for 4 or 6 hr. The migrated cells on the underside of transwells were stained with crystal violet and counted under a microscope. The quantitative results are presented as average numbers of migrated cells per transwell±S.D. of triplicates, representative of five independent experiments. *p<0.05 (Student's t test). Both mock and Ad-36 infected cells do not migrate toward 0.1% BSA (control).

FIG. 4 illustrates aspects of the invention showing the effects of Ad-36 on glucose uptake and gene expression in MCF-10A cells. Panel A is a graph Ad-36 enhances glucose uptake in dose dependent manner. Cells in 12-well plates at 80% confluence were infected with indicated dose of Ad-36. After 5 days post infection, cells were starved overnight and subjected to H³-2-deoxy-D-glucose uptake assay. The results are shown as mean±S.D. of five individual wells (*p<0.05 compared to uninfected control). Panel B is a gel showing Ad-36 increases FAS expression under starvation conditions and prolongs AKT activation after starvation. Cells were infected with Ad36 at MOI of 3.8, starved for 16 hours, then cultured in complete medium. At indicated time points, cell were lysed and subjected to immunoblotting.

FIG. 5 illustrates aspects of the invention showing Ad-36 increases AKT phosphorylation/activation and FAS expression in MCF-7 cells. Cells infected with Ad-36 at MOI 3.8 for 2 hours were starved in serum-free medium overnight and then stimulated with EGF (25 ng/ml) for the indicated time. The cells were lysed and subjected to immunoblotting analysis (Panel A). RT-qPCR analysis indicated increased FAS mRNA in Ad-36 infected cells. The mRNA levels are presented as percentages relative to 18S (Panel B).

FIG. 6 illustrates aspects of the invention showing expression of Ad-36 E4orfl enhances cell growth and induces loss of contact inhibition of MCF-10A. Vector control, Ad-2 E4orfl and Ad-36 E4orfl were transduced into MCF-10A using lentivirus. The expression of the Ad-2 E4orfl and Ad-36 E4orfl in stably transduced cells was revealed by reverse-transcription PCR (data not shown). Cell proliferation assay was performed described above for FIG. 2. Panel A is a graph showing cell numbers counted for each condition (*, p<0.05). Panel B shows representative photographs of transduced MCF-10A cells stained with crystal violet after four days in culture.

FIG. 7 illustrates aspects of the invention showing that expression of Ad-36 E4orfl gene enhances malignant potential of MCF-10A. Panel A is a graph showing expression of Ad-36 E4orfl promotes glucose uptake independent of insulin signaling. Lentivirus transduced MCF-10A cells were stimulated with 100 nM insulin 5 mins before glucose uptake analysis. Mean±S.D. of six individual wells are shown. Panel B is a graph showing triglyceride content of the cells was assayed by Oil Red O staining by isopropanol extraction. The lipid content is represented as absorbance at 510 nm S.D. of six individual wells. Panel C is a gel showing overexpression of Ad-36 E4orfl strongly increases FAS expression and AKT phosphorylation/activation. The cells were stimulated with EGF (25 ng/ml), and subjected to immunoblotting analysis. Panel D is a graph showing the increased mRNA expression of fatty acid synthase in Ad-36 E4orfl-expressing cells was confirmed by RT-PCR.

FIG. 8 shows FAS protein expression in MCF-7 cells that were infected with Ad-36 virus and treated with either Didox or Tridox. MCF-7 cells were incubated with Adv36 and a range of 10 uM/L to 100 uM/L of Didox or Tridox. Western blot of FAS protein in the samples showed that the amount of FAS decreased with Didox and Tridox treatment.

FIG. 9 shows FAS protein expression in 3T3-L preadipocyte cells maintained in MDI medium with and without Ad-36 infection and Tridox treatment. Cells incubated in MDI, with or without infection with Ad-36 had a significant increase in FAS expression. Cells infected with Ad-36 and then incubated in MDI and 10 μM of Tridox had a modest decrease in FAS expression. Cells pre-treated with Tridox and then incubated with MDI and Ad-36 had a greater decrease in FAS expression.

FIG. 10 is a graph showing that luteolin inhibits Ad-36 infection. A549 cells were pretreated with luteolin, luteolin was removed, and then the cells incubated with Ad-36. The TCID50 value was calculated and the low value in Group C suggests decreased Ad-36 virulence.

FIG. 11 is a graph showing that luteolin reduces the growth rate of MCF-10A cells infected with Ad-36. MCF-10A cells were either infected with a MOI of 0.5 or had an equivalent amount of medium added (mock infected) at day 0 for 2 hours, then moved to fresh medium containing luteolin 5 μM or an equivalent amount medium lacking luteolin. Over a time course, cells were trypsinized and counted using a hemocytometer. As illustrated, ⋄ refers to mock infected cells incubated without luteolin; □ refers to mock infected cells treated with luteolin; Δ refers to Ad-36-infected cells incubated without luteolin; and × refers to Ad-36-infected cells treated with luteolin. The cells infected with Ad-36 and exposed to luteolin had a slow growth rate, suggesting that luteolin inhibits virus-stimulated growth rate.

FIG. 12 shows Akt phosphorylation in Ad-36 infected human breast cells (MCF-10A) is reduced upon exposure to luteolin alone, metformin alone, and a combination of luteolin and metformin. Akt phophorylation is stimulated by Ad-36 as well as luteolin only, metformin only, and a combination of luteolin and metformin treatment in uninfected cells. When cells are infected with Ad-36 and treated with luteolin, metformin, or the combination of luteolin and metformin, there is a marked reduction of Akt phosphorylation. Phosphorylated Akt is a cancer marker in human cells. These results suggest that these antiviral agents can reduce the aggressiveness of cancers due to Ad-36 infection.

FIG. 13 shows a table summarizing the effect of luteolin and/or metformin at different concentrations on replication of Ad-36 in A549 cells.

### DETAILED DESCRIPTION OF THE INVENTION

It is understood that the invention is not limited to the particular methodology, protocols, devices, apparatus, materials, and reagents, etc., described herein, as these may vary. It is also to be understood that the terminology used herein is used for the purpose of describing particular aspects and embodiments only, and is not intended to limit the scope of the invention.

Unless defined otherwise, all technical and scientific terms used herein have the same meanings as commonly understood by one of ordinary skill in the art to which this invention belongs. Preferred methods, devices, and materials are described, although any methods and materials similar or equivalent to those described herein can be used in the practice or testing of the invention. All references cited herein are incorporated by reference herein in their entirety.

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary depending upon the desired properties sought to be obtained by the invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Moreover, provided immediately below is list of definitions as used herein, where certain terms related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

“AKT” refers to the serine/threonine protein kinase, also known as protein kinase B (PKB) or RAC-PK.

“CEBP” is CCAAT-enhancer binding protein.

“CPE” refers to cytopathic effect, namely degenerative changes in cells, especially in tissue culture generally associated with the multiplication of certain viruses (e.g., lipogenic adenoviruses).

“FAS” refers to fatty acid synthase.

“PI3K” refers to phosphatidylinositol 3-kinase.

“PPAR” refers to peroxisome proliferator activated receptors.

“SREBP” refers to sterol regulatory element binding protein.

“TCID50” refers to 50% tissue culture infective dose (TCID50), an endpoint dilution assay that quantifies the amount of virus required to kill 50% of infected hosts or to produce a cytopathic effect in 50% of inoculated tissue culture cells.

The term “lipogenic adenovirus” as used herein generally refers to adenoviruses that are capable of stimulating increase lipid production in cells, tissues, and/or organs by turning on the cellular machinery in infected hosts to turn on the host's production of lipogenic enzymes, lipogenic transcription factors, and glucose transport in cells, which then produce excess fatty acids and promote fat storage or oncogenic changes within the infected cells. Lipogenic adenoviruses is the same class of adenoviruses as adipogenic adenoviruses. Lipogenic adenoviruses include without limitation adenovirus type 5 (Ad-5), adenovirus type 36 (Ad-36), and adenovirus type 37 (Ad-37). As used herein, “lipogenic adenovirus particle” refers to a complete virus (also known as a virion) consisting of the viral genomic nucleic acid surrounded by a protective coat of protein called a capsid.

As used herein, the term “lipogenic adenovirus-related condition” refers to a condition, disease or disorder for which the onset of the disease or disorder is at least in part caused by lipogenic adenovirus infection or for which the symptoms of the disease or disorder are aggravated.

The term “individual” or “subject” as used herein refers to a human or a non-human animal who is or may be infected with an lipogenic adenovirus.

A “biological sample” refers to a sample of tissue or fluid from a human or animal including, but not limited to plasma, serum, spinal fluid, lymph fluid, the external sections of the skin, respiratory, intestinal and genitourinary tracts, tears, saliva, blood cells, tumors, organs, tissue and sample of in vitro cell culture constituents.

The term “antiviral agent” or “antiviral drug” refers to a chemical compound or biological material that, when administered to a subject, alone or in combination with one or more other antiviral agents, kills viruses, prevents viral entry into cells, blocks viral effects on infected cells, and/or suppresses viral replication and, hence, inhibits the capability of the virus to infect, multiply, reproduce, or cause molecular or biochemical changes within the cells. Included are derivatives and analogs of those compounds or classes of compounds specifically mentioned that also induce the desired pharmacologic effect. In particular, a therapeutic agent encompass a biological compound, a chemical compound, or a combination of biological compounds and/or chemical compounds that cause a desirable therapeutic effect.

As used herein, the terms “treating” or “treatment” refer to a reduction in the severity and/or frequency of symptoms, elimination of symptoms and/or underlying cause, reducing the likelihood of the occurrence of symptoms and/or their underlying cause, and improvement or remediation of damage. Thus, for example, the method of “treating” or “treatment” of individuals infected with a lipogenic adenovirus encompasses the treatment of infected individuals afflicted with a lipogenic adenovirus-related cancers and/or diseases.

The term “inhibition” as used herein refers to a reduction in the parameter being measured. For example, inhibition refers to a reduction in adenovirus type 36 growth, viability, infectivity, or ability to cause molecular or biochemical changes within infected cells. The amount of such reduction is measured relative to a standard (control). The term “reduction” as used herein refers to a decrease in at least around 25% relative to control, preferably at least around 50%, and most preferably at least around 75%.

The term “effective amount” or “effective dose” as used herein refers to an agent as provided herein as a nontoxic but sufficient amount of the agent to provide the desired therapeutic effect in the subject. The exact amount required will vary from subject to subject, depending on the age, weight, and general condition of the subject, the severity of the condition being treated, the judgment of the clinician, and the like. Thus, it is not possible to specify an exact “effective dose.” However, an appropriate “effective” dose in any individual case can be determined by one of ordinary skill in the art using only routine experimentation.

As used herein, the term “titration” refers to an incremental change in the dosage of an antiviral agent and/or antiviral compound such that the antiviral agent and/or antiviral compound is administered at a level that provides the desired therapeutic effect in the subject. Thus, the term “downward titration” refers to an incremental decrease in the dosage of the antiviral agent and/or antiviral compound. Thus, in addition, the term “upward titration” refers to an incremental increase in the dosage of the antiviral agent and/or antiviral compound.

As used herein, the term “nucleic acid sequence” includes oligonucleotides, nucleotides, or polynucleotides, and fragments thereof, DNA or RNA of genomic or synthetic origin, single-stranded (ss) or double-stranded (ds) molecules, peptide nucleic acid (PNA), and any DNA-like or RNA-like material, natural or synthetic in origin. Nucleic acid sequences can represent the sense or antisense strand.

As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein. These terms are used interchangeably herein to describe protein molecules that include partial or full-length proteins. Abbreviations for amino acid residues are the standard 3-letter and/or 1-letter codes used in the art to refer to one of the 20 common L-amino acids.

As used herein, the term “fragment” in the context of polypeptides or nucleic acids includes any portion of a polypeptide or nucleic acid sequence. Heterologous peptide fragments retain at least one structural or functional characteristic of the subject heterologous polypeptides. Nucleic acid sequence fragments are greater than about 60 nucleotides in length, at least about 100 nucleotides in length, at least about 1000 nucleotides in length, and at least about 10,000 nucleotides in length. Nucleic acid sequence fragments also includes probes and primers, wherein the probes or primers are at least about 8 nucleotides in length, at least about 10 nucleotides in length, at least about 12 nucleotides in length, at least about 15 nucleotides in length, at least about 18 nucleotides in length, and at least about 20 nucleotides in length. Peptide fragments can be greater than about 60 amino acids in length, at least about 100 amino acids in length, at least about 1000 amino acids in length, and at least about 10,000 amino acids in length. In addition, peptide fragments, such as for use in production or detection of antibodies, can be at least about 5 amino acids in length, at least about 8 amino acids in length, at least about 10 amino acids in length, at least about 12 amino acids in length, and at least about 15 amino acids in length.

As used herein, the terms “isolated” or “substantially pure” refers to a protein or nucleic acid (e.g., DNA, RNA, or a mixed polymer) that is substantially separated from other cellular components which naturally accompany a native human or animal sequence or protein, e.g., ribosomes, polymerases, many other human or animal genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems. Isolated or substantially purified generally refers to molecules at least 60% free, preferably at least 75% free, and most preferably at least 90% free from other components with which they are naturally associated.

As used herein, the term “immunoassay” generally refers to a test that employs antibody and antigen complexes to generate a measurable response. The term “antibody:antigen complex” and the term “immuno-complex” are used interchangeably. Immunoassays, in general, include noncompetitive immunoassays, competitive immunoassays, homogenous immunoassays, and heterogeneous immunoassays. In “competitive immunoassays,” unlabeled analyte (or antigen) in the test sample is measured by its ability to compete with labeled antigen in the immunoassay. The unlabeled antigen blocks the ability of the labeled antigen to bind because the binding site on the antibody is already occupied. In “competitive immunoassays,” the amount of antigen present in the test sample is inversely related to the amount of signal. Conversely, in “noncompetitive immunoassays,” the analyte is bound between two highly specific antibody reagents and the amount of antigen is directly proportional to the amount of signal. Immunoassays that require separation of bound antibody:antigen complexes are generally referred to as “heterogeneous immunoassays,” and immunoassays that do not require separation of antibody:antigen complexes are generally referred to as “homogeneous immunoassays.” Immunoassay methodologies are known by those of ordinary skill in the art and are appreciated to include radioimmunoassay (RIA), enzyme immunoassays (EIA), fluorescence polarization immunoassays (FPIA), microparticle enzyme immunoassays (MEIA), and chemiluminescent magnetic immunoassays (CMIA).

The term “pharmaceutically acceptable carrier” as used herein refers to compounds and compositions that are suitable for use in human or animal subjects, such as, for example, for therapeutic compositions administered for the treatment of lipogenic adenovirus-associated diseases.

The term “pharmaceutical composition” is used herein to denote a composition that can be administered to a subject, e.g., orally, parenterally, topically, by inhalation spray, intranasally, or rectally, in unit dosage formulations containing conventional non-toxic carriers, diluents, adjuvants, vehicles and the like.

The term “parenteral” as used herein, includes subcutaneous injections, intravenous, intramuscular, intracisternal injection, or infusion techniques.

Embodiments of the invention provide methods of treating lipogenic adenovirus-related conditions. In various embodiments, the method comprises administering a composition comprising an effective dose of an antiviral agent to a subject having a lipogenic adenovirus-related condition. In some aspects of the invention, administration of the antiviral agent prevents or reduces lipogenic adenovirus proliferation and/or action in the subject. In certain aspects of the invention, administration of the antiviral agent reduces or eliminates one or more symptoms of the lipogenic adenovirus-related condition.

In some aspects of the invention, the method further comprises administering a composition comprising an effective dose of a therapeutic agent known to treat the lipogenic adenovirus-related condition in conjunction with the composition comprising an effective dose of an antiviral agent. In some aspects of the invention, administration of the therapeutic agent reduces or eliminates one or more symptoms of the lipogenic adenovirus-related condition. In certain aspects of the invention, administration of the antiviral agent and the therapeutic agent reduces or eliminates one or more symptoms of the lipogenic adenovirus-related condition more efficiently than administration of either the antiviral agent or the therapeutic agent alone.

In certain aspects of the invention, the method comprises identifying a subject having a lipogenic adenovirus-related condition, wherein the subject is infected with a lipogenic adenovirus. In some aspects of the invention, identifying a subject having a lipogenic adenovirus-related condition comprises determining whether a nucleic acid sequence specific to the lipogenic adenovirus is present in the biological sample. In some aspects of the invention, identifying a subject having a lipogenic adenovirus-related condition comprises determining whether antibodies specific to the lipogenic adenovirus are present in the biological sample. In certain aspects of the invention, identifying a subject having a lipogenic adenovirus-related condition comprises determining whether lipogenic adenovirus proteins and/or lipogenic adenovirus particles are present in the biological sample.

In certain aspects of the invention, the subject is a human. In other aspects of the invention, the subject is mammalian or avian.

In certain aspects of the invention, the lipogenic adenovirus-related condition treated is cancer, obesity, diabetes, pancreatic dysfunction, liver disease, liver dysfunction, cirrhosis, muscle dysfunction, pulmonary dysfunction, brain and nervous system dysfunction, and/or adrenal dysfunction. In some aspects of the invention, the lipogenic adenovirus-related condition is diabetes mellitus. In certain aspects of the invention, the disease is diabetes mellitus type I. In other aspects of the invention, the disease is diabetes mellitus type II. In some aspects of the invention, the lipogenic adenovirus-related condition is Alzheimer's disease.

In some aspects of the invention, the lipogenic adenovirus-related condition is cancer. In certain aspects of the invention, wherein the lipogenic adenovirus-related condition is cancer, the cancer is one or more of prostate cancer, breast cancer, uterine cancer, ovarian cancer, colon cancer, lung cancer, kidney cancer, and pancreatic cancer. In some aspects, the lipogenic adenovirus-related condition comprises a cancer other than the cancers listed above.

In certain aspects of the invention, lipogenic adenoviruses cause human cancers. Without being limited to any particular mechanism, lipogenic adenoviruses can act to alter expression of genes in the host that allow unregulated cell growth to occur (e.g., oncogenes). For example, hereditary breast cancer has been linked to germline mutations in high penetrance susceptibility genes such as BRCA1, BRCA2, CHEK 2, TP53 or PTEN. In certain aspects of the invention, lipogenic adenovirus infections facilitate cancer in these genetically susceptible individuals. In some aspects of the invention, lipogenic adenoviruses may contribute to spontaneous oncogenesis by inducing expression of various oncogenes or suppressing expression of tumor suppressor genes of the host. For example, in some aspects of the invention, lipogenic adenovirus infection alter expression of genes that facilitate the development of cancer such as DNA-dependent protein kinase, fatty acid binding protein, mTOR, p16, p53, PDZ protein, phosphatidylinositol 3-kinase, PML, thymidine kinase, and Zip kinase. In certain aspects of the invention, such genes are influenced by the adenovirus E4 region, including the E4orfl gene. For example, in some aspects of the invention, the E4 region influences expression of DNA-dependent protein kinase, p53, PDZ protein, phosphatidylinositol 3-kinase, PML, thymidine kinase, and Zip kinase. In certain aspects of the invention, the Ad-36 E4orfl gene is involved in producing obesity by a direct effect on adipocyte metabolism.

In certain aspects of the invention, the lipogenic adenovirus comprises one or more of adenovirus type 5, adenovirus type 36, and adenovirus type 37. In some aspects of the invention, the lipogenic adenovirus comprises adenovirus type 36.

In certain aspects of the invention, the antiviral agent comprises one or more of a variety of antiviral agents. For example, in some aspects of the invention, the antiviral agent comprises one or more of a ribonucleotide reductase inhibitor, a nucleoside analog, a nucleotide analog, a protease inhibitor, an antisense drug, a ribozyme, a trace mineral binder, an antioxidant, an AMP-activated protein kinase (AMPK) activator, and/or an interferon drug. For example, in some aspects of the invention, the antiviral agent comprises one or more of Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lamivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, Zidovudine, conjugated Linoleic acid, Echinacea, Elder berry, Garlic, Hyssop, Kahalalide F, Licorice Root, Lycoris radiate, St. John's Wort, Uncaria tomentoas, Zostrix, metformin, luteolin, conjugated linoleic acid, N-acetylcysteine, monolaurin, alpha lipoic acid, melatonin, and any combination thereof. In some aspects of the invention, antiviral agents other than those listed above are appropriate as known in the art or determined empirically.

In certain aspects of the invention, the antiviral agent is be administered intranasally, orally, or by injection intravenously, intramuscularly, subcutaneously, and/or peritoneally.

In some aspects of the invention, dosing schemes are developed to assist in selection of effective dosage for the antiviral agent and/or the therapeutic agent. In some aspects of the invention, the effective dose of the antiviral agent is in a range of about 0.5 mg/m² to about 3000 mg/m², or about 1.0 mg/m² to about 6.0 mg/m², or about 3.0 mg/m² to about 15 mg/m², or about 10 mg/m² to about 50 mg/m², or about 30 mg/m² to about 100 mg/m², or about 100 mg/m² to about 500 mg/m², or about 200 mg/m² to about 1000 mg/m², or about 800 mg/m² to about 2000 mg/m², or about 1800 mg/m² to about 3000 mg/m². In some aspects of the invention, the effective dose of the antiviral agent is at least about 50 μg, or at least about 100 μg, or at least about 250 μg, or at least about 500 μg, or at least about 5 mg, or at least about 20 mg, or at least about 50 mg, or at least about 100 mg. In other aspects of the invention, other ranges are appropriate as known in the art or as determined empirically. In some aspects of the invention, the effective dose of the therapeutic agent is in a range of about 0.5 mg/m² to about 3000 mg/m², or about 1.0 mg/m² to about 6.0 mg/m², or about 3.0 mg/m² to about 15 mg/m², or about 10 mg/m² to about 50 mg/m², or about 30 mg/m² to about 100 mg/m², or about 100 mg/m² to about 500 mg/m², or about 200 mg/m² to about 1000 mg/m², or about 800 mg/m² to about 2000 mg/m², or about 1800 mg/m² to about 3000 mg/m². In some aspects of the invention, the effective dose of the therapeutic agent is at least about 50 μg, or at least about 100 μg, or at least about 250 μg, or at least about 500 μg, or at least about 5 mg, or at least about 20 mg, or at least about 50 mg, or at least about 100 mg. In some aspects of the invention, other ranges are appropriate as known in the art or as determined empirically.

In some aspects of the invention, the therapeutic agent administered in conjunction with the composition comprising the antiviral agent comprises a chemotherapeutic agent. In certain aspects of the invention, the therapeutic agent is one or more of an alkylating agent, an antimetabolite, an anthracycline, a plant alkaloid, a topoisomerase inhibitor, a cytotoxic antibiotic, a targeted therapeutic or a hormone. For example, in certain aspects of the invention, the therapeutic agent comprises one or more of cisplatin, carboplatin, oxaliplatin, mechlorethamine, cyclophosphamide, chlorambucin, ifosfamide, azathioprine, mercaptopurine, thioguanine, fludarabine, pentostatin, gemcitabine, cladribine, vincristine, vinblastine, vinorelbine, vindesine, podophyllotoxin, etoposide, teniposide, paclitaxel, docetaxel, irinotecan, topotecan, amsacrine, etoposide, etoposide phosphate, teniposide, actinomycin, aclarubicin, bleomycin, dactinomycin, daunorubicin, doxorubicin, epirubicin, idarubicin, mitomycin, valrubicin, plicomycin, mitozantrone, tamoxifen, anastrozole, letrozole, fulvestrant, capecitabine, trastuzumab or metformin. In some aspects of the invention, chemotherapeutic agents other than those listed above are employed as appropriate.

Alkylating agents, antimetabolites, anthracyclines, plant alkaloids, and topoisomerase inhibitors generally exert an affect by altering cell division or DNA synthesis.

In certain aspects of the invention, alkylating agents act to alkylate nucleophilic functional groups under the conditions present in cells. In some aspects of the invention, alkylating agents include cisplatin, carboplatin, oxaliplatin, mechlorethamine, cyclophosphamide, chlorambucin, and ifosfamide.

Anti-metabolites are analogs of purine or pyrimidine, which are the building blocks of DNA. In some aspects of the invention, anti-metabolites include azathioprine, mercaptopurine, thioguanine, fludarabine, pentostatin, gemcitabine and cladribine. In some aspects of the invention, the anti-metabolite may be a pro-drug. For example, in some aspects, the pro-drug may be capecitabine, which is enzymatically converted to 5-fluorouracil intracellularly, which inhibits DNA synthesis and slows growth of tumor cells.

Plant alkaloids are generally derived from plants and, in some aspects of the invention, block cell division by preventing microtubule function. Microtubules are vital for cell division; without them, cell division cannot occur. In certain aspects of the invention, plant alkaloids are vinca alkaloids or taxanes. In some aspects, vinca alkaloids bind to specific sites on tubulin, and inhibit assembly of tubulin into microtubules. In certain aspects of the invention, vinca alkaloids include Vincristine, Vinblastine, Vinorelbine and Vindesine. In some aspects of the invention, the plant alkaloid is podophyllotoxin. In certain aspects of the invention, the therapeutic agent is a cytostatic drug. In some aspects, the cytostatic drug is etoposide or teniposide, which prevent DNA replication initiation and the replication of cellular DNA. Taxanes enhance stability of microtubules, preventing the separation of chromosomes. In some aspects, the chemotherapeutic agent is a taxane. In certain aspects of the invention, the taxanes include paclitaxel or docetaxel.

Topoisomerases are enzymes that maintain the topology of DNA intracellularly. Topoisomerase inhibitors generally interfere with transcription and replication of DNA by upsetting proper DNA supercoiling. In some aspects of the invention, topoisomerase inhibitors include irinotecan, topotecan, amsacrine, etoposide, etoposide phosphate or teniposide.

Targeted therapeutics are agents that directly target a molecular abnormality in certain types of cancer, rather than interfering with DNA processes. In some aspects of the invention, targeted therapeutics include monoclonal antibodies and tyrosine kinase inhibitors. For example, in some aspects of the invention, the target therapeutic is trastuzumab.

Cytotoxic antibiotics generally interfere with DNA replication and protein synthesis. In some aspects of the invention, cytotoxic antibiotics include actinomycin, aclarubicin, bleomycin, dactinomycin, daunorubicin, doxorubicin, epirubicin, idarubicin, mitomycin, valrubicin, plicomycin and mitozantrone.

In some aspects of the invention, hormone treatment involves administration of therapeutics that regulate hormone production and hormone activity. In some aspects of the invention, hormone treatment can kill cancer cells, make cancer cells grow more slowly, or stop them from growing. In certain aspects, hormone therapy as a cancer treatment may involve taking medications that interfere with the activity of the hormone or stop the production of the hormones, or it may involve surgically removing a gland that is producing the hormones. In some aspects of the invention, hormone treatment includes Tamoxifen (Nolvadex®), anastrozole (Arimidex®), letrozole (Femara®), and fulvestrant (Faslodex®).

In some aspects of the invention, the antiviral agent and/or the therapeutic agent are attached to an antibody (or antibody fragment or antibody derivative), wherein the antibody targets administration of the antiviral agent and/or therapeutic agent to particular cells in the subject. For example, in some aspects of the invention, wherein the lipogenic adenovirus-related disease is cancer, the antibody attached to the antiviral agent and/or the therapeutic agent specifically binds to an extracellular cell protein highly expressed on the cancer cells. In certain aspects of the invention, targeting of the antiviral agent and/or the therapeutic agent to the cells of interest increases the efficacy of the antiviral agent and/or the therapeutic agent.

In certain aspects of the invention, administration of the antiviral agent prevents or reduces lipogenic adenovirus proliferation and/or action. Lipogenic adenovirus action generally refers to the downstream molecular or biochemical effects of lipogenic adenovirus infection within an infected cell. In some aspects, the effect of lipogenic adenovirus infection occurs generally in cells infected by the adipogenic adenovirus. In certain aspects, effects of lipogenic adenovirus infection can occur in cells adjacent to or near cells infected with by the adipogenic adenovirus. In some aspects of the invention, the downstream effects of lipogenic adenovirus infection include one or more of increased glucose transport, increased expression of FAS, and increased synthesis of fatty acids.

In certain aspects of the invention, the downstream effects of lipogenic adenovirus infection include one or more of increased expression and/or activation of one or more of lipogenic enzymes and/or one or more of lipogenic transcription factors. Lipogenic transcription factors generally regulate expression of proteins involved in lipogenesis. In some aspects of the invention, lipogenesis can occur in one or more of pre-adipocyte cells, adipocyte cells, pre-cancerous cells and cancer cells. In certain aspects of the invention, lipogenesis can occur in other types of cells than those listed above. In some aspects of the invention, the lipogenic transcription factors comprise one or more of peroxisome proliferator-activated receptor gamma (PPAR-γ), CCAAT/enhancer binding protein alpha (C/EBP-α), C/EBP-β, sterol regulatory element-binding protein 1 (SREBP-1), and carbohydrate responsive element- binding protein (ChREBP).

Also, without being limited to any one particular mechanism of action, in some aspects of the invention, administration of the composition comprising the antiviral agent inhibits and/or reduces the expression of adipocyte differentiation factors. In some aspects of the invention, adipocyte differentiation factors are lipogenic transcription factors. For example, in some aspects of the invention, administration of the composition comprising the antiviral agent inhibits and/or reduces the expression of one or more of peroxisome proliferator-activated receptor gamma (PPAR-γ), CCAAT/enhancer binding protein alpha (C/EBP-α), C/EBP-β, sterol regulatory element-binding protein 1 (SREBP-1), and carbohydrate responsive element- binding protein (ChREBP). In certain aspects of the invention, reduced the expression of adipocyte differentiation factors, such as, e.g., PPAR-γ, C/EBP-α, C/EBP-β, SREBP-1 ChREBP, down-regulates differentiation of non-adipocyte cells into adipocyte cells.

Without being limited to any one particular mechanism of action, in some aspects of the invention, administration of the composition comprising the antiviral agent inhibits and/or reduces the expression of lipogenic enzymes and/or lipogenic transcription factors. For example, administration of the composition comprising the antiviral agent inhibits and/or reduces the expression of one or more of fatty acid synthase (FAS), glycerol-3-phosphophate dehydrogenase (GPDH), lipoprotein lipase (LPL), stearoyl-CoA desaturase 1 (SCD1), carnitine palmitoyltransferase 1 (CPT1), L-type pyruvate kinase (L-PK), proteins in the phosphatidylinositol 3-kinase (PI3K) signaling pathway, and proteins in the AKT/Protein Kinase B (PKB) signaling pathway. As explained herein, in some aspects of the invention, lipogenic virus infection stimulates one or more of these pathways. In some aspects of the invention, administration of the antiviral agent inhibits the lipogenic adenovirus from stimulating the phosphatidylinositol 3 (PI3)-kinase pathway and AKT pathways, fatty acid synthase pathway and/or promoting glucose uptake into the cell.

Phosphatidylinositol 3-kinases are a family of enzymes involved in cellular functions such as cell growth, proliferation, differentiation, motility, survival and intracellular trafficking, which in turn are involved in cancer. The phosphoinositide 3-kinases, P13-kinase (PI3K), family is organized into three Classes; I, II, and III. The preferred substrate of class I, PI3-kinases is phosphoinositide(4,5)bisphosphate (PIP2). This is also a substrate for members of the PI-phospholipase C family and the product of PTEN dephosphorylation of PtdIns(3,4,5)P3. Phosphorylation of PIP2 by PI3-kinase generates PtdIns(3,4,5)P3. PtdIns(3,4,5)P3 and its 5′-dephosphorylation product, PtdIns(3,4,)P2, are second messengers that coordinate to promote cell survival, growth, protein synthesis, mitosis, and motility. Class II, PI3-kinases preferentially phosphorylates phosphatidylinositol (PI) and PtdIns(4)P to form PtdIns(3)P and PtdIns(3,4)P2, respectively. Class II, PI3-kinases also phosphorylate PtdIns(4,5)P2 in the presence of phosphatidylserine (PS). Class III, PI-kinases preferentially phosphorylate phoshatidylinositol (PtdIns) to form phosphoinositol-3-P (PtdIns(3)P). PtdIns(3)P has important roles in vesicular and protein trafficking. Class III, PI3-kinase is involved in targeting lysosomal enzymes to the endocytic pathway.

Many of the cellular functions of PI3K relate to the ability of class I P1 3-kinases to activate AKT. The plecktrin homology domain of AKT binds directly to PtdIns(3,4,5)P3 and PtdIns(3,4)P2, which are produced by activated PI 3-kinase. Because PtdIns(3,4,5)P3 and PtdIns(3,4)P2 are restricted to the plasma membrane, this results in translocation of AKT to the plasma membrane. Likewise, the phosphoinositide-dependent protein kinase 1 also contains a pleckstrin homology domain that binds directly to PtdIns(3,4,5)P3 and PtdIns(3,4)P2, causing it to also translocate to the plasma membrane upon activation of PI 3-kinase. The colocalization of activated PDK1 and AKT allows AKT to become phosphorylated by PDK1, leading to partial activation of AKT. Full activation of AKT occurs upon additional phosphorylation by the TORC2 complex of the mTOR protein kinase. The PI3K/AKT signaling pathways have been shown to be required for an extremely diverse array of cellular activities, most notably cellular proliferation and survival.

Activated Akt plays a key role in mediating signals for cell growth, cell survival (anti-apoptotic), cell-cycle progression, differentiation, transcription, translation, and glucose metabolism. Because it can block apoptosis, and thereby promote cell survival, Akt1 has been implicated as a major factor in many types of cancer. Akt regulates cellular survival and metabolism by binding and regulating many downstream effectors. Akt can be activated via the PI3-kinase signaling pathway. P13K dependent Akt activation can be regulated through the tumor suppressor PTEN, which works essentially as the opposite of PI3K. PTEN acts as a phosphastase to dephosphorylate PtdIns(3,4,5)P3 back to PtdIns(4,5)P2. This removes the membrane-localization factor from the Akt signaling pathway. Without this localization, the rate of Akt activation decreases significantly, as do the all the downstream pathways that depend on Akt for activation.

In some aspects of the invention, the prevention or reduction of lipogenic adenovirus proliferation and/or action caused by administration of the antiviral agent reduces cancer aggressiveness where the lipogenic adenovirus-related disease is cancer. In certain aspects of the invention, administration of the antiviral agent in conjunction with the therapeutic agent reduces cancer aggressiveness more efficiently that administration of either the antiviral agent or the therapeutic agent alone. Examples of cancers in which cancer aggressiveness can be reduced include, but are not limited to breast cancer and prostate cancer.

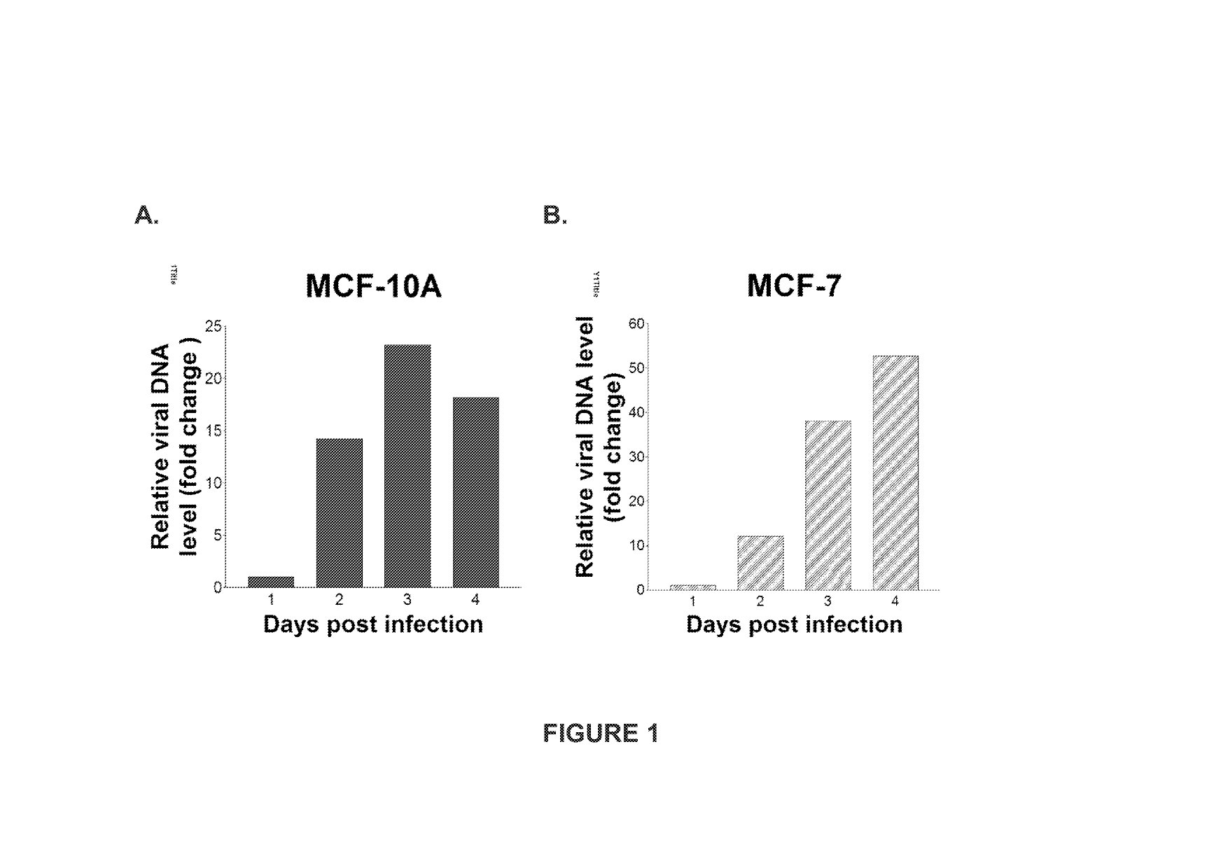

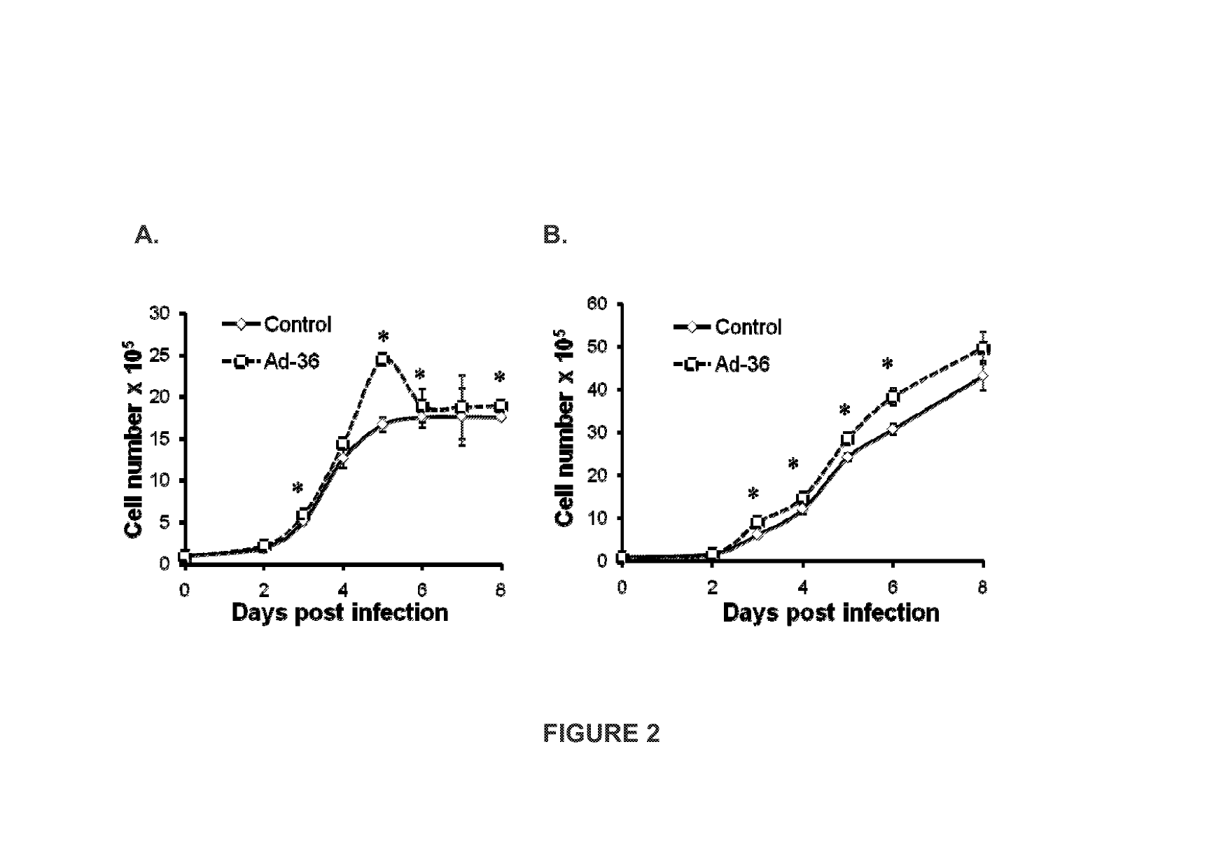

Without being limited to any one particular mechanism of action, in some aspects of the invention, lipogenic adenoviruses, such as Ad-36, promote cancer aggressiveness by increasing glucose transport into cells and increasing AKT and PI3-kinase enzyme activity. The AKT and PI3-kinase pathways regulate glucose transport and apoptosis, both of which are important for cancer. Without being bound by theory, glucose transport generally plays a role in aggressiveness because a metastatic cell must have an ability to take up glucose and protect itself from anoikis. Anoikis, cell death caused by detachment from basement membrane, is caused by starvation. Nature 461, 109-113 (2009). Cancer cells must sustain their energy production and remain well fed to survive detachment from their normal habitat By modulating the AKT and PI3-kinase pathways, lipogenic adenoviruses allows the infected cell to import glucose to escape anoikis, both of which would promote cell survival. Therefore, in some aspects of the invention, administration of an effective amount of an antiviral agent, which blocks Ad-36 from stimulating the PI3-kinase pathway, AKT pathway and glucose transport into the cell, prevents or reduces cancer aggressiveness. In certain aspects of the invention, as illustrated in FIGS. 1 and 2, Ad-36 infection is increased in breast cancer patients and enhances malignant potential of breast cells. For instance, the Examples show that 39% of breast cancer patients were infected with Ad-36, compared to only 16% of non-cancer patients. Additionally, studies have shown that over 35%, and as many as 50%, of prostate cancers are correlated with Ad-36 infection, as described in U.S. Pat. Nos. 7,442,511; 7,507,418; 7,745,110; and 7,910,310. Therefore, in certain aspects of the invention, the lipogenic adenovirus infection rate is increased in individuals having cancer.

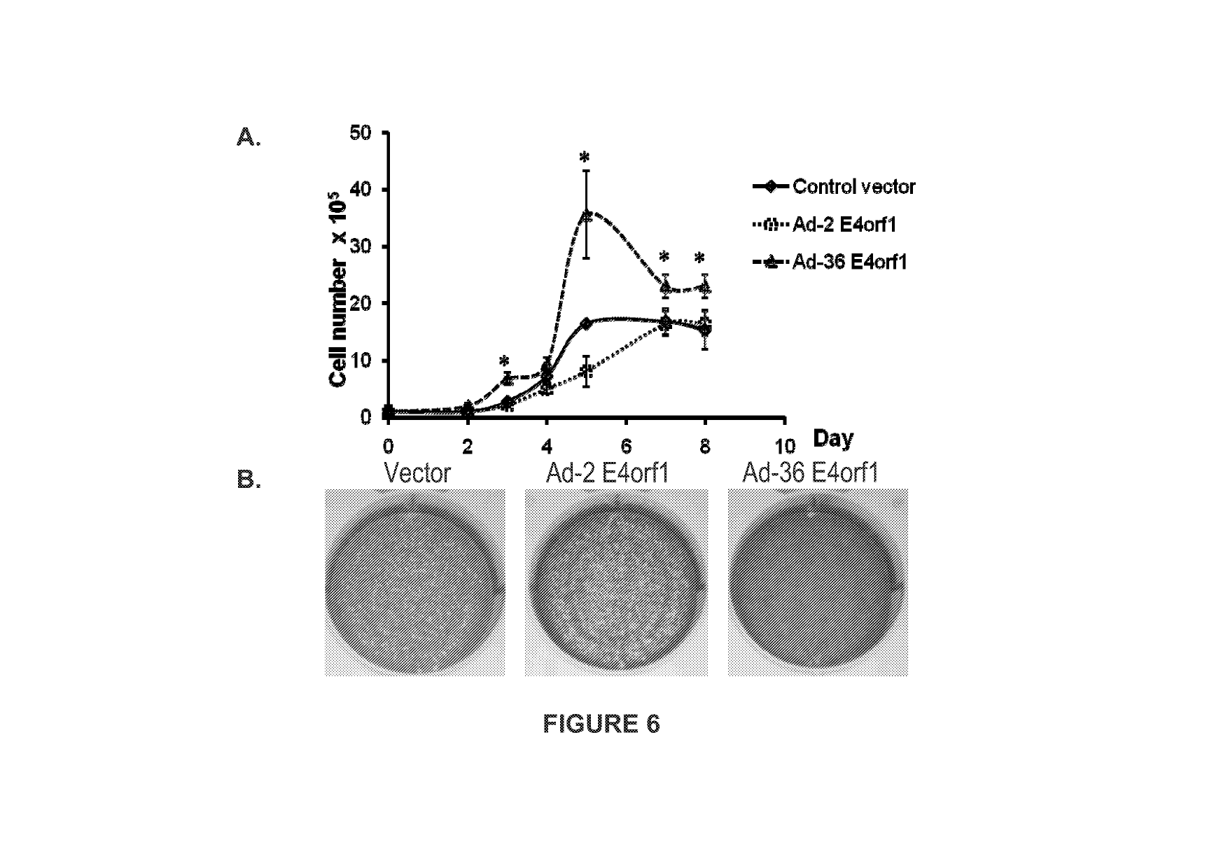

In some aspects of the invention, lipogenic adenovirus infection also produces malignant changes in cells and these changes can be ameliorated, reduced or inhibited by administration of the antiviral, as described herein. In particular, in some aspects, malignant breast cancer cells infected with Ad-36 have increased glucose uptake, increased activation of the PI3K and AKT signaling pathways, and increased fatty acid synthase (FAS) expression, a key de novo lipogenesis enzyme previously identified as a marker of aggressive breast cancer, as illustrated in FIG. 6. Further, in certain aspects of the invention, expression of the Ad-36 E4orfl gene in malignant breast cancer cells is strongly enhanced cell proliferation, causes E4orfl-expressing cells to overcome cell contact inhibition, and promotes AKT activation and glucose uptake. Therefore, in certain aspects of the invention, infection with lipogenic adenovirus enhances the malignant potential of a cell.

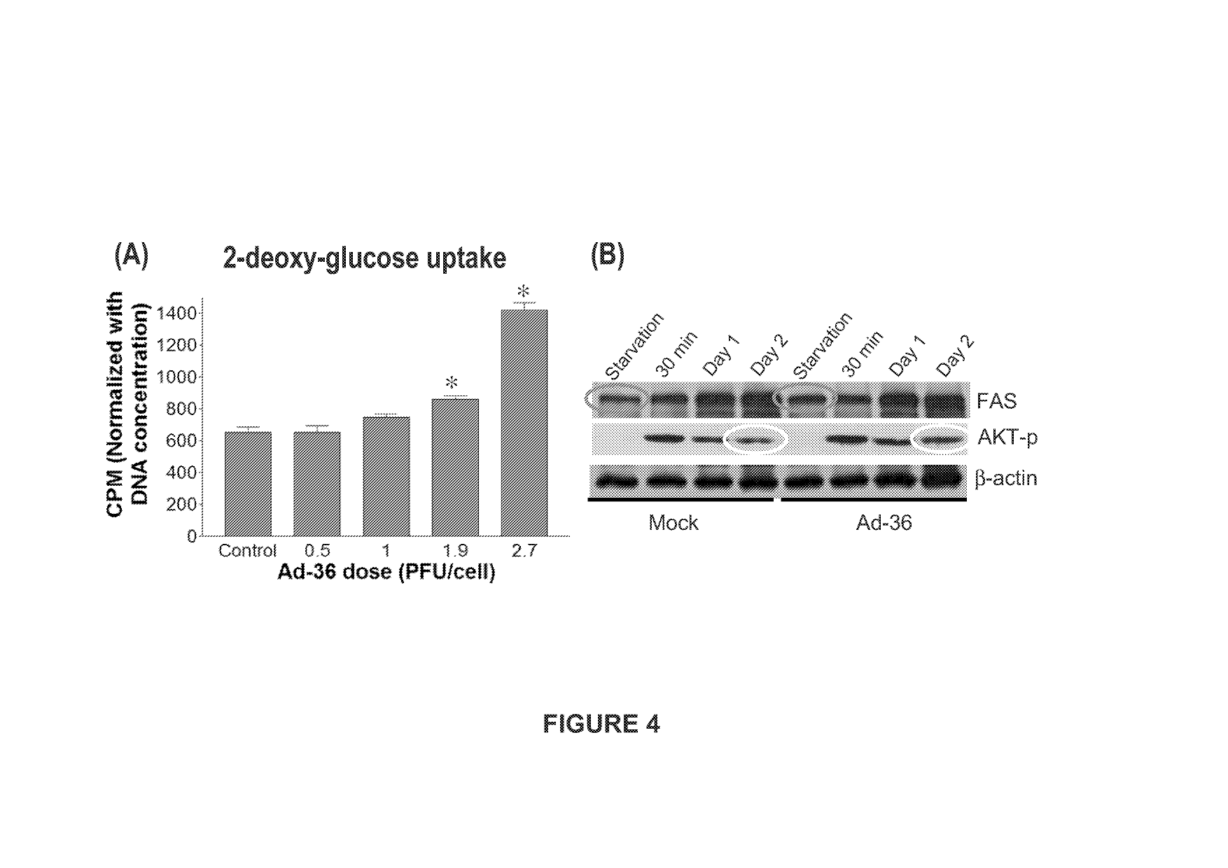

FAS is a marker for aggressive cancer and has been found in breast, prostate, and colon cancers. In certain aspects of the invention, FAS expression and lipid accumulation are also increased in cancer cells infected with lipogenic adenovirus. In particular, in some aspects, malignant breast cancer cells infected with Ad-36 have increased fat accumulation as illustrated in FIG. 4. In certain aspects, cells may use the stored lipids to grow and divide, and, as such, accumulated intracellular lipids in cancer cells can promote cancer in an individual having lipogenic adenovirus-associated cancer. Ad-36 has been shown to stimulate FAS, which makes fat from glucose within cells, and FAS is associated with malignant cancers, such as breast, colon, and prostate. Treatment of fast growing tumors is particularly important in prostate and colon cancers because the primary tumor can block off the urethra or the GI tract, respectively. Studies have shown that administration of an antiviral agent in vitro prevents lipid accumulation in fat cells. See, Rathod, et al., 2007, Int. J. Obes., 31(1): 78-86. Without being bound by theory, in some aspects of the invention, excess fat produced by FAS is a mechanism promoting cancer growth and aggressiveness in individuals having lipogenic adenovirus-associated cancer, which may be inhibited by administration of the antiviral agent. In certain aspects if the invention, antiviral agents are used to treat lipogenic adenovirus-associated cancer or reduce cancer progression by blocking lipogenic adenovirus-induced fat accumulation within the cancer cells, thus depriving them of a source of nutrients.

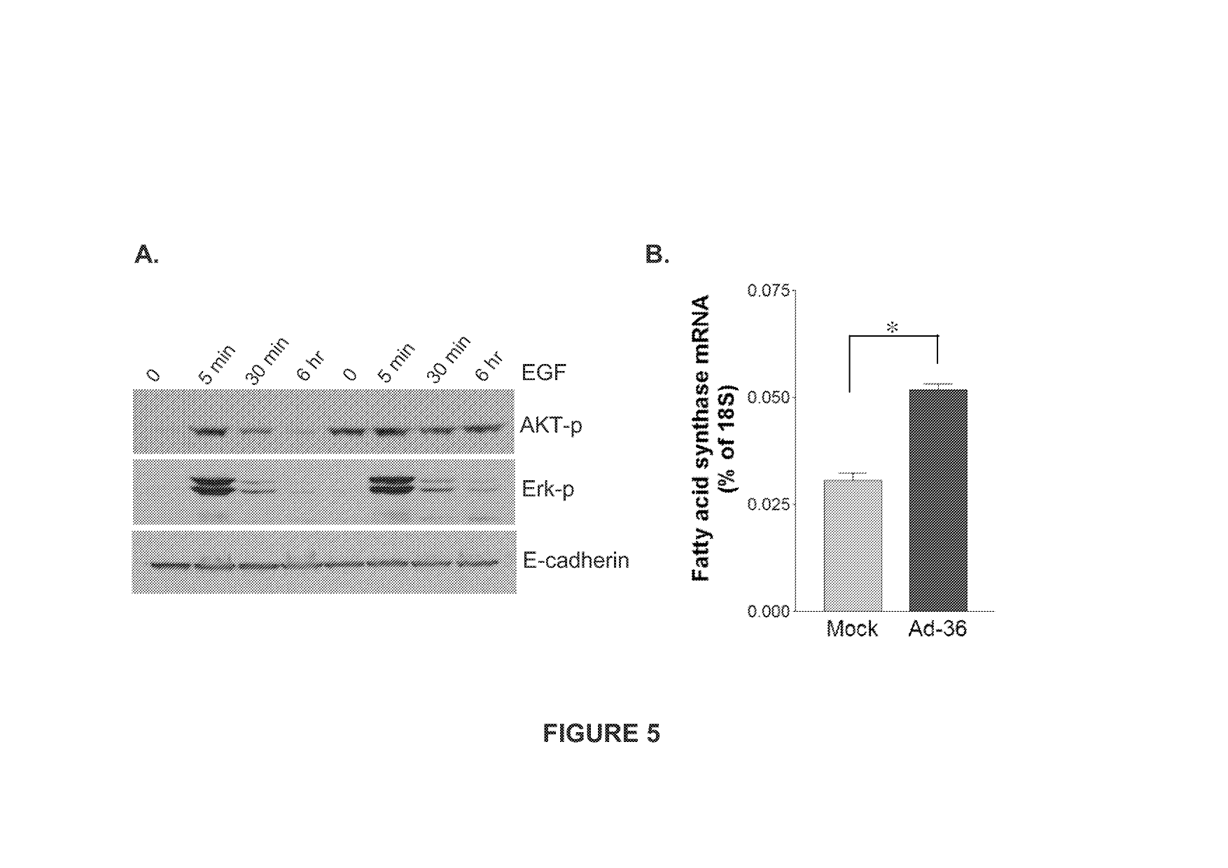

In some aspects of the invention, lipogenic adenovirus infection cause a loss of cell contact inhibition. In particular, in some aspects of the invention, benign breast cells infected with Ad-36 show a loss of contact inhibition as illustrated in FIG. 5. Loss of contact inhibition contributes to cancer growth because normal cells arrest growth and division when they come into contact with other cells, whereas cancer cells do not. If contact inhibition is decreased, the cells continue growing even though they are in contact with other cells. Thus, in some aspects of the invention, administration of an antiviral agent is used to treat cancer or cancer progression in an individual infected with a lipogenic adenovirus and having cancer by restoring contact inhibition to the cancerous lipogenic adenovirus-infected cells, therefore constraining tumor growth.

Lipogenic adenovirus infection is shown herein to promote development and aggressiveness of cancer. In certain aspects of the invention, administration of an effective dose of an antiviral agent to an individual infected with a lipogenic adenovirus, such as Ad-36, prevents or reduces cancer aggressiveness in the individual. In certain aspects of the invention, antiviral treatment also improves the efficacy of chemotherapy and other treatments by weakening the cancer such that it is more susceptible to chemotherapy.

The following aspects of the invention relate to the embodiments set forth above.

### Antiviral Compositions

Certain aspects of the invention relate to administering a composition including an effective dose of an antiviral compound or a combination of antiviral compounds to prevent, reduce the incidence of and/or ameliorate proliferation and/or viral effects on cells and/or replication of a lipogenic adenovirus. In particular, for example, the antiviral are administered to a subject afflicted with a lipogenic adenovirus related cancer and/or other lipogenic adenovirus related disease. Lipogenic adenoviruses include adenoviruses that are capable of stimulating an increase lipid production in cells, tissues, and/or organs by facilitating increased glucose transport into cells and expression and/or activation of lipogenic enzymes and/or lipogenic transcription factors, which in turn produce excess fatty acids and promote fat storage. The lipogenic adenoviruses of the invention include, for example, Ad-5, Ad-36, and Ad-37. Some aspects of the invention relate to therapeutic compositions for use in reducing or eliminating one or more symptoms of a lipogenic adenovirus-related conditions. Compositions of the invention may be produced using methods of formulation well known in the art. Further, compositions of the invention may be administered to a subject in need thereof using standard modes of administration known in the art.

In certain aspects, the therapeutic compositions include an effective dose of an antiviral agent, wherein administration of the antiviral agent prevents or reduces lipogenic adenovirus proliferation and/or action; an effective dose of a therapeutic agent, wherein the therapeutic agent known to treat the lipogenic adenovirus-related condition; and a pharmaceutically acceptable carrier.

In some aspects, the invention relates to use of an antiviral agent in preparation of a medicament for treatment of a lipogenic adenovirus-related condition, characterized in that the antiviral agent prevents or reduces lipogenic adenovirus proliferation and/or action. In certain aspects, the medicament further comprises a therapeutic agent, wherein the therapeutic agent is known to treat the lipogenic adenovirus-related condition.

Some aspects of the invention relate to methods of making a therapeutic composition for treatment of a subject having a lipogenic adenovirus-related condition. In certain aspects, the method comprises selecting an antiviral agent, wherein the antiviral agent prevents or reduces lipogenic adenovirus proliferation and/or action; and selecting a pharmaceutically acceptable carrier. In some aspects of the invention, the method further includes selecting a therapeutic agent, wherein the therapeutic agent is known to treat the lipogenic adenovirus-related condition. Selection of the therapeutic agent will depend on the nature of the lipogenic adenovirus-related condition(s) that the subject has. In some aspects of the invention, the therapeutic composition includes more than one antiviral agent. In certain aspects of the invention, the therapeutic composition includes more than one therapeutic agent. In certain aspects, wherein the therapeutic composition includes more than one therapeutic agent, the therapeutic agents may be for treatment of different lipogenic adenovirus-related conditions.

In some aspects of the invention, lipogenic adenovirus-related diseases include, inter alia, diabetes mellitus, hypertension, hyperlipoproteinemia, cardiac disease such as atherosclerotic disease and congestive heart failure, pulmonary diseases such as sleep apnea and asthma, cerebrovascular accidents, cancers such as breast, uterus colon and prostate cancer, gall bladder disease such as stones and infection, toxemia during pregnancy, risks during surgery, gout, decreased fertility, degenerative arthritis, and early mortality. In certain aspects of the invention, the antiviral composition prevents the production and/or acceleration of lipogenic adenovirus-related diseases.

While not limiting the invention to a particular mechanism, in certain aspects of the invention, lipogenic adenovirus infection stimulates lipogenic enzymes that increase fat deposition in adipose tissues and cause differentiation of adult stem cells in adipose tissue into adipocytes. In some aspects of the invention, lipogenic enzymes are expressed or over-expressed in lipogenic adenovirus-infected cells, such as fatty acid synthase (FAS), glycerol-3-phosphodehydrogenase, lipoprotein lipase (LPL), SREBP-1, SCD1, CPT 1, PPAR-gamma, AKT, PI3K signaling pathway and L-type pyruvate kinase. In certain aspects of the invention, these lipogenic enzymes are responsible for increased glucose transport and the formation of excess fatty acids and promote fat storage within the cells of multiple organs. In some aspects, these enzymes also reduce fatty acid oxidation. Therefore, in certain aspects of the invention, the effective amount of the antiviral agent inhibits or reduces the expression of lipogenic enzymes such as FAS, glycerol-3-phosphodehydrogenase, lipoprotein lipase, AKT, PI3K signaling pathway, SCD1, CPT 1, L-type pyruvate kinase, malic enzyme, glucose 6 phosphate dehydrogenase, DGAT1, S14, 6 phosphogluconic dehydrogenase, acetyl coA carboxylase (CBX) and citrate cleavage enzyme. In certain aspects of the invention, lipogenic enzymes are expressed or over-expressed in lipogenic adenovirus-infected cells.

Antiviral agents or antiviral drugs are a class of compounds used specifically to prevent or treat viral infections. Generally, an antiviral agent kills viruses, suppresses viral replication, blocks viral actions, and/or prevents viral infection of cells, thereby inhibiting the ability of the virus to multiply, reproduce, or alter normal host cell biochemistry and physiology. In some aspects of the invention, antiviral agents are useful in the early stages of some lipogenic adenovirus infections, or to prevent reoccurrences or reactivation in chronic lipogenic adenovirus, or to block chronic effects of lipogenic adenovirus action on normal host cell biochemistry and physiology. In certain aspects of the invention, antiviral agents may exert their effects only during a certain stage of lipogenic adenovirus replication, or may act both early and to affect chronic infections. In certain aspects of the invention, antiviral agents are designed to target and disable viral proteins (i.e., prevent protein function), viral DNA, or viral RNA. In some aspects of the invention, viral targets of antiviral agents are generally unrelated to any proteins, protein domains, DNA, or RNA in humans or animals to be treated with the antiviral agents in order to reduce unacceptable side effects.

Antiviral agents have been developed to disable viral replication at different steps of the viral life cycle including, but not limited to, viral entry into the host cell, replication of the viral genome, activation of viral protein, release of new virus particles, and inhibition of viral action on normal host cell biochemistry or physiology. For example, in some aspects of the invention, the antiviral agent may inhibit the ability of a virus to enter a host cell by preventing the lipogenic adenovirus from binding to receptors on the host cell that are required for entry, or blocking the virus uncoating process inside the host cell such that the lipogenic adenovirus cannot release its contents. In additional aspects of the invention, antiviral agents may target the processes that synthesize viral components after a lipogenic adenovirus invades a host cell (e.g., adenovirus DNA polymerase). There are several classes of antiviral agents that block viral synthesis including, but not limited to, ribonucleotide reductase inhibitors, nucleotide analogs, nucleoside analogs, antisense drugs, ribozymes, and protease inhibitors. Finally, antiviral agents such as interferon drugs may function to prevent the release of newly packaged virus particles from the infected host cell thereby blocking the final stage of the viral life cycle. In some aspects of the invention, antiviral agents that block lipogenic adenovirus replication at any of the stages in the adenovirus life cycle or block the ability of the adenovirus to alter normal host cell biochemistry or physiology may be effective treatments for preventing lipogenic adenovirus-related cancer, or other lipogenic adenovirus- related diseases.

In different aspects of the invention, antiviral agents act in different ways to inhibit or prevent proliferation and/or action of lipogenic adenoviruses in the treatment of lipogenic adenovirus-related cancers and/or diseases. In some aspects of the invention, the antiviral agent prevents the viral infection altogether by preventing lipogenic adenovirus attachment to the host cell, thereby preventing the occurrence of the lipogenic adenovirus related cancer and/or disease in the individual. In other aspects of the invention, an antiviral agent prevents the lipogenic adenovirus from being active within the host's cells. In some aspects of the invention, such antiviral agents act by one or more of several mechanisms and/or at one of several steps, including: (i) an antiviral agent may prevent the lipogenic adenovirus DNA from entering into the nucleus of the host cell thereby preventing the lipogenic adenovirus from causing the cancers and/or diseases associated with infection; (ii) an antiviral agent may block the viral lipogenic adenovirus from activating the host's DNA to increase production of FAS or glucose transporters; (iii) antiviral agents may block the transcription of lipogenic adenovirus DNA to RNA or translation of RNA to protein; (iv) antiviral agents may block the effects of the lipogenic adenovirus proteins that are made by the early genes of the virus, such as E4orfl or E1A; and/or (v) antiviral agents may prevent the lipogenic adenovirus-mediated inhibition of host cell and body defense mechanisms, thereby allowing the lipogenic adenovirus infected host cell to die or be destroyed.

Treatment with the antiviral agent Cidofovir has been shown to block the lipogenic effect of Ad-36 in preadipocytes in tissue culture. Rathod et al., 2007, “Viral mRNA expression but not DNA replication is required for lipogenic effect of human adenovirus Ad-36 in preadipocytes,” Int. J. Obes., 31:78-86. As FAS has been shown to be the mechanism of production of fatty acids within the cell, treatment with Cidofovir may block the production of

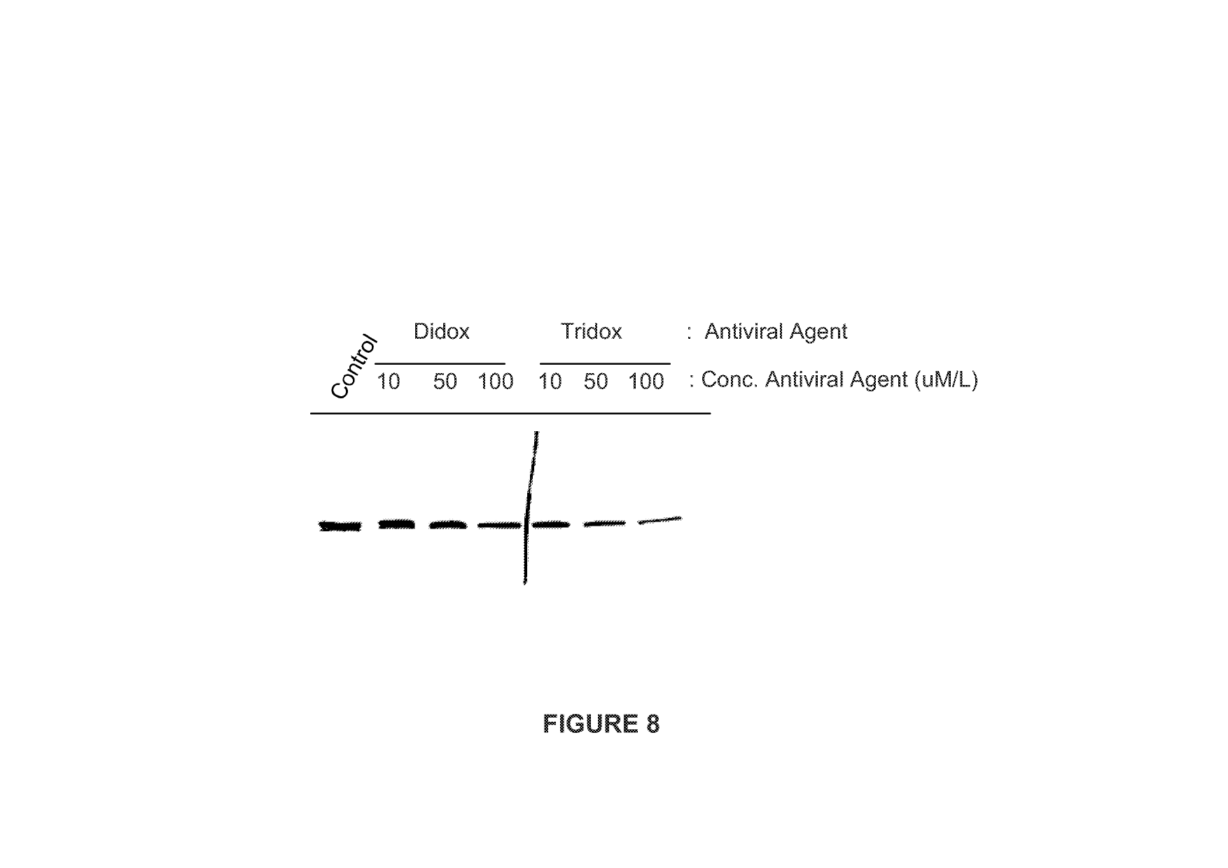

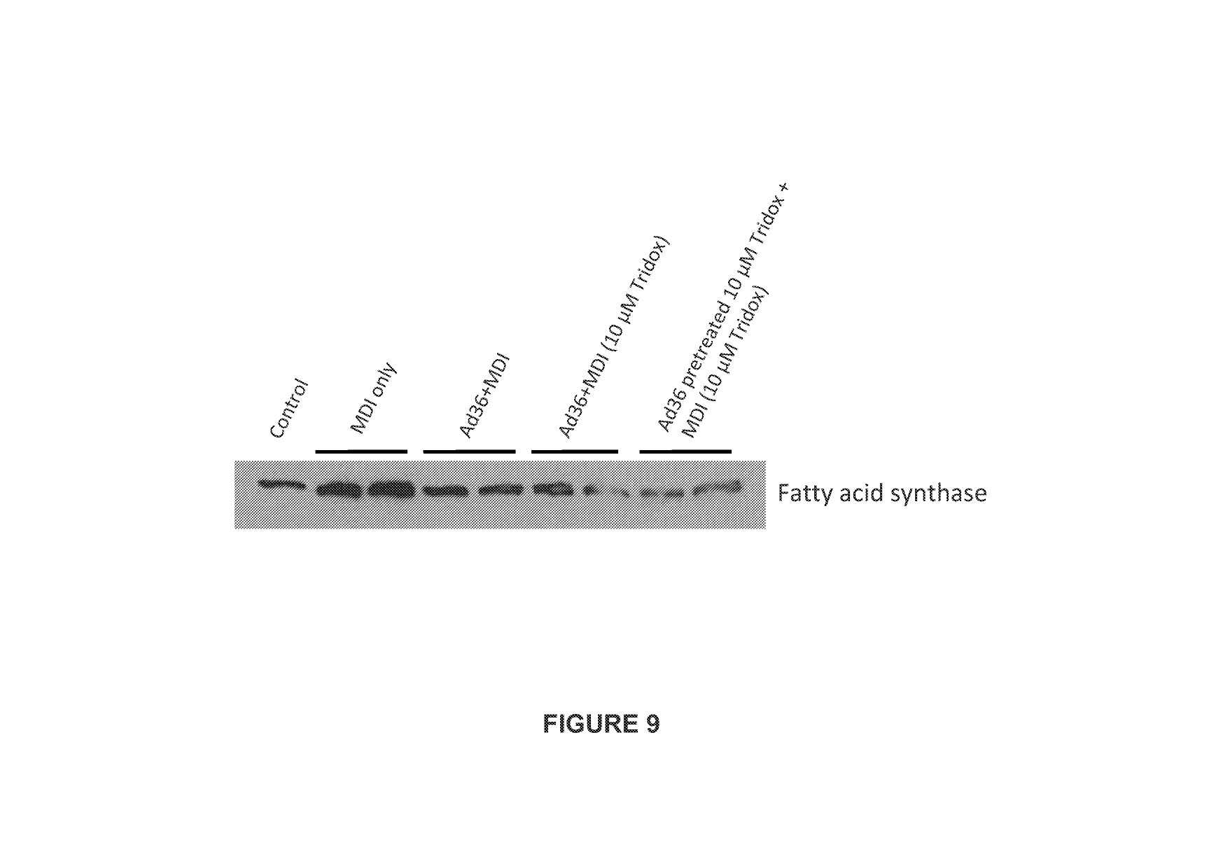

FAS. In aspects of the invention, treatment with an antiviral compound decreases the expression level of FAS in cancer cells. For example, as illustrated in FIG. 8, in some aspects of the invention, administration of Didox (3,4 dihydrobezohydoxamic acid) or Tridox (3,4,5 trihydroxybenzamidoxime) to human breast cancer cells, such as, for example, MCF-7 cells, infected with Ad-36 decreases in the protein level of FAS in the cells. In other aspects of the invention, as illustrated in FIG. 9, FAS protein in 3T3-L1 preadiopcyte cells is decreased as the dosage of Didox or Tridox is increased.

In certain aspects of the invention, the antiviral agent includes without limitation nucleoside analogs, nucleotide analogs, and/or ribonucleotide reductase inhibitors. Antiviral agents are well known in the art. A number of pharmaceutical agents have been shown to have antiviral effects, which may be used in combination with the antiviral agents of the invention. In certain aspects of the invention, the antiviral agent includes, without limitation, one or more of Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lamivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, and Zidovudine. In some aspects of the invention, another pharmaceutical agent that is not classically thought of as an antiviral agent is metformin. In other aspects, other pharmaceutical agents may be appropriate for use as antiviral agents as known in the art or determined empirically.

In addition to pharmaceutical agents, there are a number of herbal agents, dietary supplements and other alternative medicine agents that have antiviral effects and can used in combination with the antiviral agents of the invention. For example, in some aspects of the invention, non-prescription antiviral agents include, but are not limited to, one or more of: conjugated linoleic acid, Echinacea, Elder berry, garlic, Hyssop, Kahalalide F, Licorice Root, Lycoris radiate, St. John's wort, Uncaria tomentoas, Zostrix, luteolin, nicotine, caffeine, N-Acetylcysteine, monolaurin, alpha lipoic acid and melatonin. In some aspects, the antiviral agent is not nicotine or caffeine or garlic or elderberry.

Administration of the RAGE fusion proteins of the present invention may employ various routes. Thus, administration of the RAGE fusion protein of the present invention may employ intraperitoneal (IP) injection. Alternatively, the RAGE fusion protein may be administered orally, intranasally, or as an aerosol. In another aspect of the invention, administration is intravenous (IV). In some aspects, administration is subcutaneously. In other aspects, administration is intra-arterial. In some aspects, administration is sublingual. Also, administration may employ a time-release capsule. In yet another embodiment, administration may be transrectal, as by a suppository or the like. For example, subcutaneous administration may be useful to treat chronic disorders when the self-administration is desirable.

In some aspects of the invention, antiviral agents are administered to the subject intranasally, orally, or by injection intravenously, intramuscularly, subcutaneously or peritoneally. In certain aspects of the invention, the antiviral is administered in a single dose or in several steps in order to cause and maintain optimal treatment of viral infection and viral diseases in the subject being treated. For example, in some aspects of the invention, administration is once daily, twice daily, three times daily, or with other periodicity as known in the art or determined empirically. In some aspects of the invention, the dosage regimen uses an upward titration or, alternatively, a downward titration. Appropriate dosing of the antiviral agent is within the skill of medical practitioners and others skilled in the art, and will depend on several factors including the age of the subject treated, body weight, and the like.

Pharmaceutically acceptable carriers may comprise any of the standard pharmaceutically accepted carriers known in the art. The carrier may comprise a diluent. In one aspect, the pharmaceutical carrier may be a liquid and the antiviral agent, or the antiviral agent and the therapeutic agent, may be in the form of a solution. In another embodiment, the pharmaceutically acceptable carrier may be a solid in the form of a powder, a lyophilized powder, or a tablet. In some aspects, the pharmaceutical carrier may be a gel, suppository, or cream. In certain aspects, the carrier may comprise a liposome, a microcapsule, a polymer encapsulated cell, or a virus. Thus, the term pharmaceutically acceptable carrier encompasses, but is not limited to, any of the standard pharmaceutically accepted carriers, such as water, alcohols, phosphate buffered saline solution, sugars (e.g., sucrose or mannitol), oils or emulsions such as oil/water emulsions or a trigyceride emulsion, various types of wetting agents, tablets, coated tablets and capsules.

Compositions of the invention may be in various forms. The compositions may be in the form of a sterile injectable solution in a non-toxic parenterally acceptable solvent or vehicle. The compositions may be in the form of a sterile lyophilized powder for injection upon reconstitution with a diluent. The diluent can be water for injection, bacteriostatic water for injection, or sterile saline. The compositions for injection may also be in the form of a oleaginous suspension, which can be formulated according to the known methods using suitable dispersing or wetting agents and suspending agents. The compositions of the invention may also be in the form of oil-in-water emulsions or aqueous suspensions. The compositions of the invention may also contain antimicrobial preservatives, such as benzyl alcohol and parabens; surfactants to reduce aggregation, such as Polysorbate 80, poloxomer, or other surfactants known in the art; and/or other additives, such as a sugar(s) or saline, to adjust the osmotic pressure of the composition to be similar to human blood.

The compositions of the invention may optionally include one or more surfactants. The surfactant can be cationic, anionic, nonionic, or amphoteric. A wide variety of conventional surfactants can be used. Surfactants can include, without limitation, polyoxyethylenesorbitans, polyoxyethylenesorbitan monolaurate, polysorbate-20, such as Tween-20™, polysorbate-80, hydroxycellulose, and genapol, vitamin E-TPGS and lecithins or lecithin constituents. Combinations of various surfactants can be used if desired.

The compositions of the invention optionally may optionally include one or more excipients. The excipient may play a role in efficacy, e.g., delivery of the antiviral agent of the composition, and/or for improved subject compliance, e.g., improved appearance and/or taste of the composition. The excipients can be lubricants, disintegrants, fillers (diluents), binders, suspending agents, humectants, emulsifying/solubilizing agents, desiccants, coating agents, chelating agents, buffering agents, antimicrobial preservatives and acidifying agents, among others. Lubricants can include, without limitation, calcium stearate, magnesium stearate, magnesium trisilicate, sodium stearyl fumarate, stearic acid, zinc stearate. Disintegrants can include, without limitation, alginic acid, carboxymethylcellulose calcium NF, carboxymethylcellulose sodium, cellulose, croscarmellose sodium, crospovidone, microcrystalline cellulose, and sodium starch glycolate. Diluents can include, without limitation, calcium phosphate dibasic anhydrous, calcium phosphate dibasic dihydrate, calcium phosphate tribasic, cellulose powder, lactose, magnesium carbonate, and microcrystalline cellulose. Binders can include, without limitation, alginic acid, carboxymethylcellulose sodium, isomalt, microcrystalline cellulose, and povidone K30. Suspending agents can include, without limitation, alginic acid, carboxymethylcellulose, silicon dioxide, and sodium alginate. Humectants can include, without limitation, glycerin and xylitol. Emulsifying/solubilizing agents can include, without limitation, sodium stearate and stearic acid. Desiccants can include, without limitation, calcium chloride and silicon dioxide. Coating agents can include, without limitation, carboxymethylcellulose, copovidone, and titanium dioxide. Buffering agents can include, without limitation, potassium citrate, potassium phosphate monobasic, sodium acetate, and sodium phosphate mono and dibasic. Antimicrobial preservatives can include, without limitation, benzoic acid, chlorhexidine gluconate 20% USP, potassium benzoate, potassium sorbate, sorbic acid, and sodium benzoate. Acidifying agents can include, without limitation, citric acid, fumaric acid, malic acid and tartaric acid.

The compositions of the invention optionally may optionally include vitamins and dietary minerals. Vitamins administered in the composition may include, but are not limited to, vitamin B (including B vitamins B1, B2, B3, B5, B6, B7, B9, and B12), vitamin C, vitamin E, vitamin A, vitamin D, vitamin K, and derivatives thereof. Additionally, the compositions of the invention may include adenine, adenylic acid, essential fatty acids, riboflavin, biotin, flavin, anthranilic acid, adenylthiomethylpentose, folic acid, carnitine, flavonoids, niacin, and S-methylmethionine. Dietary minerals administered in the composition may include, but are not limited to, calcium, chloride, magnesium, phosphorous, potassium, sodium, cobalt, copper, manganese, and zinc.

Buffers may be useful for, among other purposes, manipulation of the total pH of the composition. A variety of buffers known in the art optionally may be used in the composition of the invention, such as various salts of organic or inorganic acids, bases, or amino acids, and including various forms of citrate, phosphate, tartrate, succinate, adipate, maleate, lactate, acetate, bicarbonate, or carbonate ions. The pH of the formulation changes according to the amount of buffer used. Depending upon the dosage form, it may alternatively be advantageous to use buffers in different concentrations or to use other additives to adjust the pH of the formulation to encompass other ranges.

Additional useful additives are readily determined by those of skill in the art, according to particular compositions. Once such particularly useful additional substance is sodium chloride, which is useful for adjusting the osmolarity of the formulations to achieve the desired resulting osmolarity.

### Immunoanalytical Detection Methods

In some aspects of the invention, antibodies reactive to lipogenic adenoviruses and lipogenic virus biomarker proteins may be employed for the detection of lipogenic adenovirus and biomarker proteins in a subject. Exemplary screening immunoanalytical techniques include without limitation, standard virus neutralization assay techniques or enzyme immunoassay techniques well known in the art.

Techniques for raising and purifying antibodies against these lipogenic adenoviruses or fragments thereof (e.g., fiber protein, hexon protein, or fragments thereof), or other proteins (or fragments thereof) from these viruses for use in these immunoassay techniques may be prepared by conventional techniques are well known in the art. With regards to human adenovirus 36, the full genomic sequence of the virus is set forth in SEQ ID NO:42, and is readily usable for these purposes, as are the fiber gene and protein sequences (SEQ ID Nos. 38 and 39) and hexon gene and protein sequences (SEQ ID Nos. 40 and 41). See also, Arnold, et al., 2010, Virus Res., 149(2): 152-161. In certain aspects of the invention, such antibodies can be used to bind lipogenic adenovirus virus or lipogenic adenovirus proteins from solution as well as react with these proteins on Western or immunoblots or polyacrylamide gels. In some aspects of the invention, the fiber protein sequences set forth in Table 1 below are be employed to generate antibodies reactive to lipogenic adenoviruses or are used to detect lipogenic adenovirus antibodies (e.g., in a serum neutralization assay).

TABLE 1
Adenovirus fiber protein peptide sequences for use
in antibody production.*

| | Amino Acid | | |
|---|---|---|---|
| SEQ | Start | End | |
| ID | Posi- | Posi- | |
| NO | tion | tion | Sequence |
| 1 | 11 | 17 | FNPVYPY |
| 2 | 24 | 35 | NIPFLTPPFVSS |
| 3 | 41 | 55 | FPPGVLSLKLADPIA |
| 4 | 57 | 73 | ANGNVSLKVGGGLTVEQ |
| 5 | 75 | 88 | SGKLSVDTKAPLQV |
| 6 | 113 | 121 | AGHGLAVVT |
| 7 | 126 | 138 | SLPSLVGTLVVLT |
| 8 | 189 | 195 | PSPNCKV |
| 9 | 201 | 232 | SKLTLALTKCGSQILATVSLLVVTGKYAIISD |
| 10 | 235 | 254 | NPKQFSIKLLFNDKGVLLSD |
| 11 | 275 | 281 | YKEAVGF |
| 12 | 316 | 329 | LGGEVYQPGFIVVK |
| 13 | 336 | 342 | ANCAYSI |
| 14 | 348 | 359 | WGKVYKDPIPYD |
| 15 | | | VETARDSKLT |
| 16 | | | LGGEVYQPGFIVVK |
| 17 | | | WGKVYKDPIPYD |
| 18 | | | GTGSSAHG |

*Peptide sequences correspond to the amino acid sequence for the fiber protein as set forth in Arnold, et al., 2010, Virus Res., 149(2): 152-161.

In other aspects of the invention, antibodies are used to detect the presence of lipogenic adenovirus or lipogenic adenovirus proteins in a biological sample, using immunocytochemical techniques. In certain aspects of the invention, methods for detecting lipogenic adenovirus or lipogenic adenovirus proteins include methods well known in the art such as enzyme linked immunosorbent assays (ELISA), radioimmunoassay (RIA), immunoradiometric assays (IRMA) and immunoenzymatic assays (IEMA), including sandwich assays using monoclonal and/or polyclonal antibodies.

In some aspects of the invention, lipogenic adenovirus proteins or fragments thereof are used to detect the presence of antibodies in biological samples obtained from individuals known, or suspected, to be infected with a lipogenic adenovirus and/or having a lipogenic adenovirus-related disease. Methods for detecting such antibodies include methods well known in the art such as, for example, enzyme immunoassay techniques or virus neutralization assays, amongst others. For example, in some aspects of the invention, a standard virus neutralization assay is used to identify the presence of antibodies reactive to a lipogenic adenovirus in a biological sample such as a serum sample obtained from a subject. In certain aspects of the invention, protein sequences such as those set forth in Table 1 are used in enzyme immunoassay techniques to detect antibodies reactive thereto in a sample from a subject.

### Nucleic Acid Detection Methods

The lipogenic adenoviruses and biomarkers may be detected by nucleic acid detection techniques (including, e.g., PCR and non-PCR techniques). The nucleic acid probe hybridization assay techniques used in these methods of the invention will be standard techniques (optionally after amplification of DNA or RNA extracted from a sample of blood, other body fluid, feces, tissue or organ) using nucleic acid probes (and primers if amplification is employed) made available by the lipogenic adenoviruses identified and made available by the invention. The sequences of nucleic acids characteristic of the lipogenic adenoviruses can be determined by standard techniques once the viruses are conventionally isolated, and probes and primers that are specific for the viruses and that provide the basis for nucleic acid probes and primers that can be used in nucleic acid based assays for the viruses are prepared using conventional techniques on the basis of the sequences.

In some aspects of the invention, screening involves amplification of the relevant lipogenic adenovirus sequences. In some aspects of the invention, the screening method involves a non-PCR based strategy. Such screening methods include two-step label amplification methodologies that are well known in the art. Both PCR and non-PCR based screening strategies can detect target sequences with a high level of sensitivity.

Certain aspects of the invention relates to target amplification. Here, the target nucleic acid sequence is amplified with polymerase. One specific method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes.

Quantitative amplification methods (e.g., quantitative PCR or quantitative linear amplification) can be used to quantify the amount of target nucleic acids. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Amplifications may be monitored in “real time.”

In general, quantitative amplification is based on the monitoring of the signal (e.g., fluorescence of a probe) representing copies of the template in cycles of an amplification (e.g.,

PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., Proc. Natl. Acad. Sci. USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer (Nature Biotech. 14:303-309 (1996)), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in the hairpin (closed) conformation, the fluorescence of the donor fluorophore is quenched. When employed in PCR, the molecular beacon probe, which hybridizes to one of the strands of the PCR product, is in the open conformation and fluorescence is detected, and the probes that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of a nucleic acid are also known. For example, some methodologies employ one or more probe oligonucleotides that are structured such that a change in fluorescence is generated when the oligonucleotide(s) is hybridized to a target nucleic acid. For example, one such method involves a dual fluorophore approach that exploits fluorescence resonance energy transfer (FRET), e.g., LightCycler™ hybridization probes, where two oligonucleotide probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux probes™).

In some aspects of the invention, intercalating agents that produce a signal when intercalated in double stranded DNA may be used. Exemplary agents include SYBR GREEN™ and SYBR GOLD™. Because these agents are not template-specific, it is assumed that the signal is generated based on template-specific amplification. This can be confirmed by monitoring signal as a function of temperature because melting point of template sequences will generally be much higher than, for example, primer-dimers, etc.

In certain aspects of the invention, the following primers may be employed for PCR amplification of the nucleic acid sequence encoding the Ad-36 hexon protein:

SEQ ID NO: 19:
5′-ggtggacaaccacccactac-3′ (Forward primer)
SEQ ID NO: 20:
5′- tggacaaccacccactacaa-3′ (Forward primer)
SEQ ID NO: 21:
5′-cagcggagcttgtatcttcc-3′ (Reverse primer)

In some aspects of the invention, nested PCR may be used to detect Ad-36 DNA in biological samples. Four primers were designed to unique regions of the Ad-36 fiber protein gene for use in a nested PCR assay for detection of viral DNA, which are as follows:

outer forward primer:
(SEQ ID NO: 22)
5′-gtctggaaaactgagtgtggata-3′,
outer reverse primer:
(SEQ ID NO: 23)
5′-atccaaaatcaaatgtaatagagt-3′,
inner forward primer:
(SEQ ID NO: 24)
5′-ttaactggaaaaggaataggta-3′,
inner reverse primer:
(SEQ ID NO: 25)
5′-ggtgttgttggttggcttaggata-3′.

In certain aspects of the invention, the following primers may be employed for detecting Ad-36 fiber protein using SYBR green method:

(SEQ ID NO: 29)
Forward: 5′-taagccaaccaacaacacca-3′
(SEQ ID NO: 30)
Reverse: 5′-tgcacaattggcatcagttt-3′
(SEQ ID NO: 31)
Forward: 5′-ggccatggtttagcagttgt-3′
(SEQ ID NO: 32)
Reverse: 5′-gtccaaagggtgcgtgtatc-3′
(SEQ ID NO: 33)
Forward: 5′-ttaactggaaaaggaataggta-3′
(SEQ ID NO: 34)
Reverse: 5′-ggtgttgttggttggcttaggata-3′

In some aspects of the invention, the following primers may be employed for detecting Ad-36 fiber protein using the Taqman™ method:

Forward:
(SEQ ID NO: 35)
5′-caatgggaatgtctcactcaaggt-3′
Reverse:
(SEQ ID NO: 36)
5′-agggtgccttagtatccacact-3′
(SEQ ID NO: 37)
5′-cagttttccagactgttgttct-3′

When the probes are used to detect the presence of the lipogenic adenovirus nucleic acid sequences or biomarker nucleic acid sequences, nucleic acid may be first isolated from the biological sample. The sample nucleic acid may be prepared in various ways known in the art, to facilitate detection of the target sequence, e.g., denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques well known in the art.

Analyte nucleic acid and probe are incubated under conditions which promote stable hybrid formation of the target sequence in the analyte. The region of the probes which is used to bind to the analyte can be made completely complementary to the targeted region of the lipogenic adenovirus of interest, and in particular the Ad-36 fiber coat protein or hexon protein. For example, the following probes may be used to detect the nucleic acid encoding Ad-36 fiber coat protein:

SEQ ID NO: 26: 5′-agttgaaacagcaagagactcaaag-3′
SEQ ID NO: 27: 5′-ggtactggatcaagtgcacatggag-3′
SEQ ID NO: 28: 5′-ttgaaacagcaagagactcaaagctaac-3′

High stringency conditions may be desirable in order to prevent false positives. However, conditions of high stringency are used only if the probes are complementary to regions of the lipogenic adenovirus. The stringency of hybridization is determined by a number of factors during hybridization and during the washing procedure, including temperature, ionic strength, base composition, probe length, and concentration of formamide.

Detection, if any, of the resulting hybrid is usually accomplished by the use of labeled probes. Alternatively, however, the probe may be unlabeled, but may be detectable by specific binding with a ligand which is labeled, either directly or indirectly. Suitable labels, and method for labeling probes and ligands are well known in the art, and include, for example, radioactive labels which may be incorporated by known methods (e.g., nick translation, random priming or kinasing), biotin, fluorescent groups, chemiluminescent groups (e.g., dioxetanes) enzymes, antibodies, gold nanoparticles and the like. Variations of this basic scheme are known in the art, and include those variations that facilitate separation of the hybrids to be detected from extraneous materials and/or that amplify the signal from the labeled moiety.

As noted above, non-PCR based screening assays are also contemplated by this invention. For example, one such procedure hybridizes a nucleic acid probe (or analog such as a methyl phosphonate backbone replacing the normal phosphodiester) to the low level DNA target. In this procedure, the probe may have an enzyme covalently linked thereto such that the covalent linkage does not interfere with the specificity of the hybridization. The enzyme-probe-conjugate-target nucleic acid complex can then be isolated away from the free probe conjugate and a substrate is added for enzyme detection. Enzymatic activity can be observed, for example, as a change in color development or luminescent output resulting in about a 10³ to about a 10⁶ increase in sensitivity.

Two-step label amplification methodologies are known in the art. These assays work on the principle that a small ligand (such as digioxigenin, biotin, or the like) is attached to a nucleic acid probe capable of specific binding the adenovirus sequence region of interest. In one example, the small ligand attached to the nucleic acid probe is specifically recognized by an antibody-enzyme conjugate. In some aspects of the invention, digioexigenin is attached to the nucleic acid probe. Hybridization is detected by an antibody-alkaline phosphatase conjugate which turns over a chemiluminescent substrate. In a second example, the small ligand is recognized by a second ligand-enzyme conjugate that is capable of specifically complexing to the first ligand. In certain aspects of the invention, biotin-avidin type interactions are used.

It is also contemplated within aspects of the invention that the nucleic acid probe assays of the invention will employ a cocktail of nucleic acid probes capable of detecting various species of adenoviruses. Thus, in one example to detect the presence of Ad-36, Ad-37 and/or Ad-5, for example, in a biological sample, more than one probe complementary of the targeted regions of interest in the various types of adenovirus may be employed.

As the skilled will understand, more than one strain of lipogenic adenovirus may be tested for simultaneously in an immunological or nucleic acid-based assay method for testing for virus in accordance with the invention and kits may be assembled to facilitate carrying out the methods for a particular virus or a plurality of them.

### Diagnostic Kits

Another aspect of the invention is directed to a diagnostic screening test kits containing reagents for use in the immunoassay methods described above. Typically, such a kit contains at least one lipogenic adenovirus-specific protein, such as a fiber coat-protein and/or at least one reagent that specifically binds to the lipogenic adenovirus specific protein, such as an anti-fiber coat protein antibody. Kits typically also includes directions or instructions describing how to perform the above-described diagnostic assays, and/or how to interpret the results thereby obtained. In some kits, anti-fiber coat protein antibodies are linked to an immobilized solid support and/or the fiber coat protein is immobilized on a solid support. The anti-fiber coat protein antibody or fiber-coat protein may or may not be linked to an appropriate label.

The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label may be a specific binding molecule which itself may be detectable (e.g., biotin, avidin, streptavidin, digioxigenin, maltose, oligohistidine, 2,4-dinitrobenzene, phenylarsenate, ssDNA, dsDNA, etc.).

The solid support may include, without limitation, paper, sponge materials, cellulose, wood, woven and nonwoven fabrics, glass fiber, polymeric films, preformed and microporous membranes, synthetic and modified naturally-occurring polymers, or hydrophilic inorganic powders. Further, the solid support may be a test strip and may include a support strip or handle, normally constructed from a hydrophobic plastic, and a reagent test region, containing a bibulous or a nonbibulous carrier matrix incorporating the anti-fiber coat antibodies and/or the fiber-coat protein. The carrier matrix may be an absorbent material that allows the test sample to move, in response to capillary forces, through the carrier matrix to contact the anti-fiber coat antibody and/or fiber-coat protein and produce a detectable or measurable signal, such as a color transition. The carrier matrix can be any substance capable of incorporating the anti-fiber coat protein antibody and/or fiber-coat protein, as long as the carrier matrix is substantially inert, and is porous or absorbent relative to the soluble components of the liquid test sample.

### EXAMPLES

Features and advantages of the inventive concept covered by the invention are further illustrated in the examples which follow.

As used herein, MCF-7 (HTB 22)is a malignant human breast adenocarcinoma cell line, MCF-10A (CRL 10317) is a benign human mammary epithelial cell line, 3T3-L1 (CL 173)is a mouse embryonic fibroblast-adipose like cell line, and A549 (CCL 185) is a human lung adenocarcinoma epithelial cell line. Each of these cell lines was obtained from the ATCC.

As used herein, Didox (3,4 dihydrobezohydoxamic acid) and Tridox (3,4,5 trihydroxybenzamidoxime)were obtained from Molecules for Health.

The full length sequence of the human adenovirus 36 genome is set forth in SEQ ID NO:42. The sequence of the Ad-36 E4orfl gene as used herein is also set forth in Rogers, et al., 2008, Int. J. Obes., 32(3): 397-406.

Methods described herein were performed according to procedures well known in the art.

### Example 1

### Increased Incidence of Ad-36 Infection in Breast Cancer Patients

Blood from 128 women with breast cancer was assayed for antibodies to Ad-36 by serum neutralization assay and compared to 183 non-cancer controls. Breast cancer patient samples were obtained from the Comprehensive Cancer Center of the University of Wisconsin. Patients were anonymous, and no information other than the fact that the patients had been diagnosed with breast cancer was known for the purposes of this study. Control samples were from the Clinical Nutrition Center at the University of Wisconsin, and contained an oversampling of obese women. As obese individuals have a higher incidence of Ad-36 infection, the data were normalized to reflect % obese/lean in the overall general population.

Ad-36 infection was found to be increased in breast cancer patients relative to healthy patients. Ad-36 antibodies were seen in 39% of cancer patients and in 16% (population adjusted) of non-cancer patients (p<0.001). If both positive and equivocal tests were combined, 59% of breast cancer patients were positive. These results are shown in Table 2 below.

TABLE 2
Prevalence of Ad-36 antibodies in serum of female
breast cancer patients and non-cancer controls.

| Patient Type (Number) | % Positive | % Negative |
|---|---|---|
| Breast cancer patients (128) | 39% | 61% |
| Non-cancer control patients (183) | 16% | 84% |
| (% population adjusted) | | |

### Example 2

### Ad-36 Infection Enhances Malignant Potential of Breast Cells In Vitro

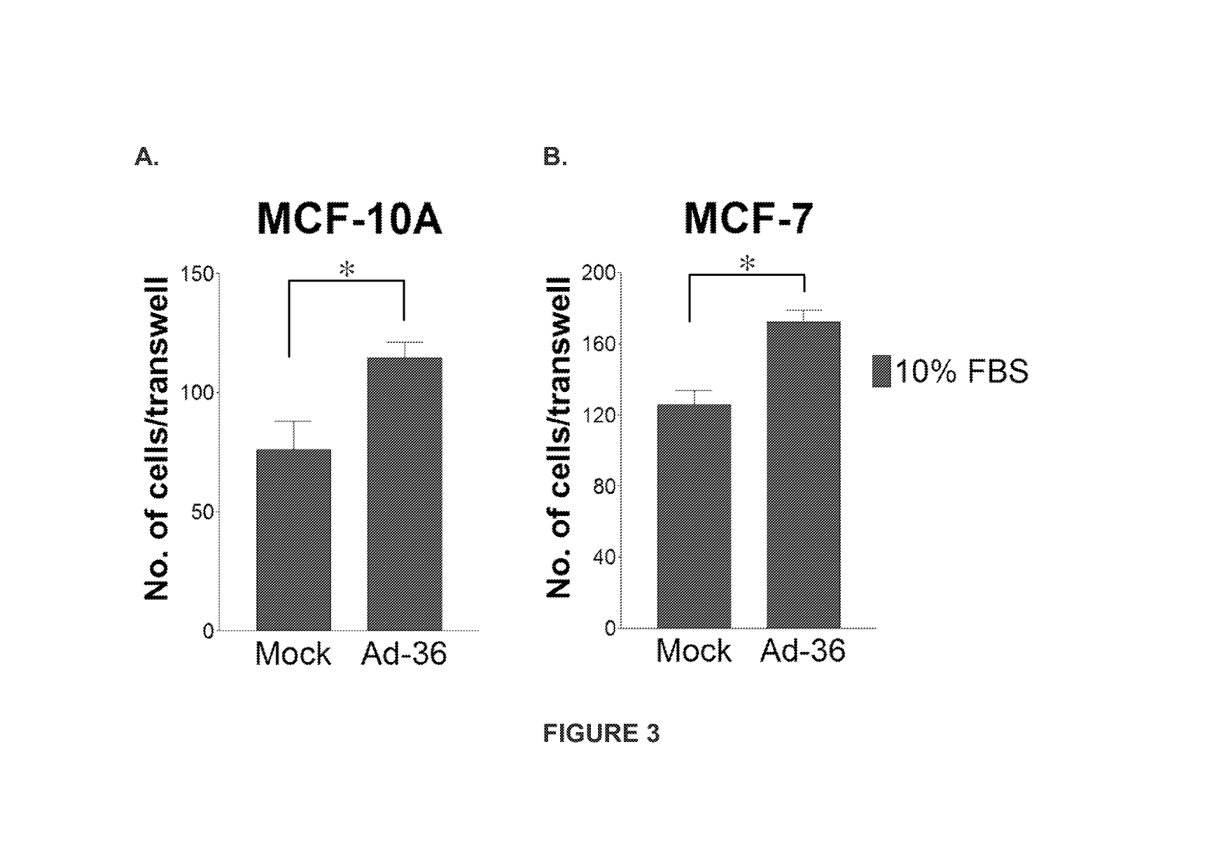

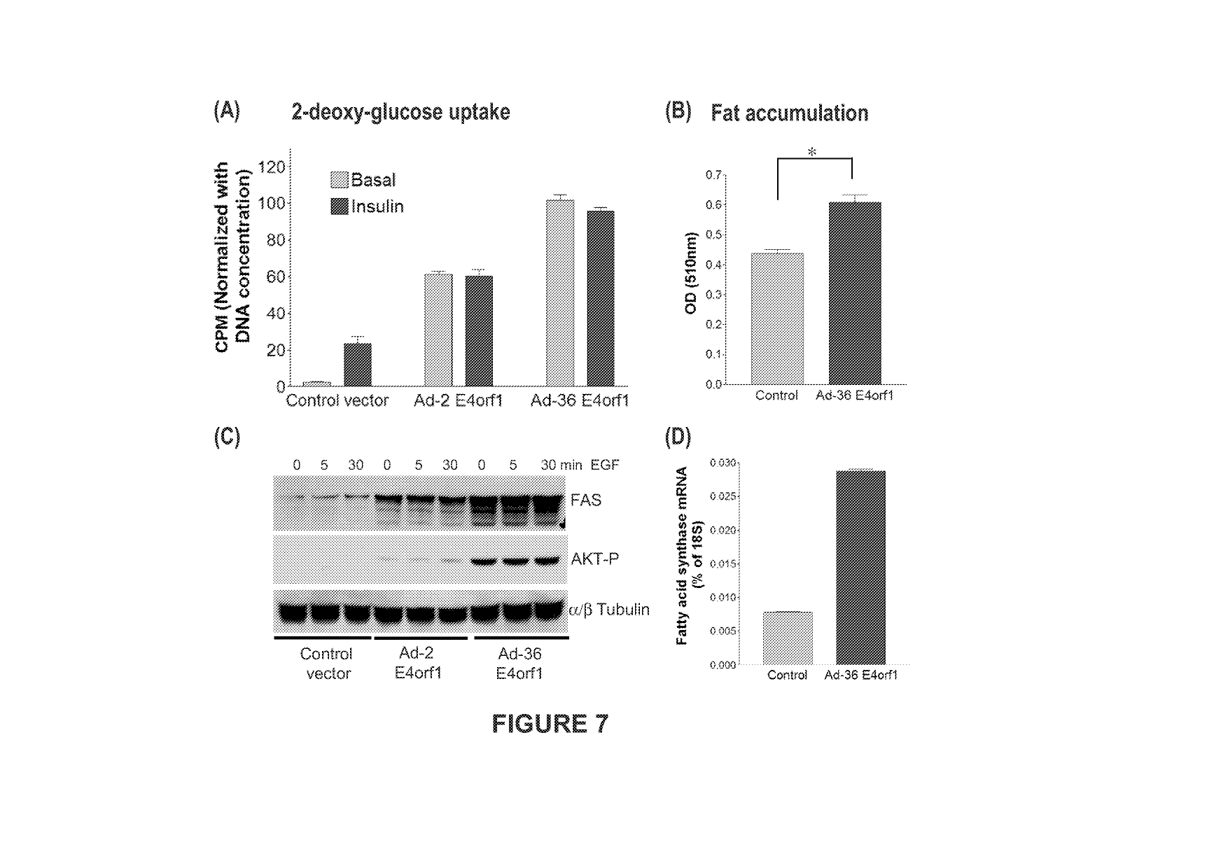

Benign (MCF-10A) and malignant (MCF-7) breast cell lines were infected with Ad-36. The Ad-36 E4orfl gene was transduced into cells via lentivirus using procedures well known in the art. See e.g., Vangipuram, et al., 2004, Obes. Res., 12(5): 770-777. The malignant characteristics of growth rate, migratory ability, glucose uptake and cancer enzyme markers were compared to uninfected cells as illustrated in FIGS. 1-7.

In vitro, Ad-36 infection significantly increased growth rate and migratory ability of both

MCF-10A and MCF-7 cells. In MCF-10A cells, Ad-36 induced robust increase in glucose uptake. Ad-36 infection increased phosphatidylinositol 3-kinase (PI3K) signaling pathways, AKT, and fatty acid synthase (FAS) expression. Ectopic over-expression of Ad36 E4orfl gene in MCF-10A cells strongly enhanced cell proliferation, overcame cell contact inhibition, promoted AKT activation, FAS expression, glucose uptake and enhanced lipid accumulation.

### Example 3

### Effects of Didox and Tridox on FAS Protein in MCF-7 Cells Infected with Ad-36

Human breast cancer cells (MCF-7) were infected with Ad-36 virus and treated with antiviral compounds Didox or Tridox. MCF-7 cells were incubated with Ad-36 and a range of about 10 μM/L to about 100 μM/L of Didox or Tridox. The control for the experiments was MCF-7 cells infected with Ad-36 that were not treated with an antiviral compound. Fifty micrograms of protein was loaded per lane on an SDS-PAGE gel, followed by Western blot analysis detecting FAS.

As illustrated in FIG. 8, FAS protein expression was found to decrease with about 50 μM and about 100 μM Didox treatment and about 10 μM, about 50 μM and about 100 μM Tridox treatment. Thus, antiviral treatment of Ad-36-infected breast cancer cells was capable of lowering expression of the cancer biomarker FAS.

### Example 4

### Effect of Tridox on FAS Protein Expression in 3T3-L1 Cells

The effects of Tridox on FAS protein expression were evaluated in 3T3-L preadipocyte cells. Control cells were maintained without exposure to Ad-36 or to the differentiation cocktail of methyl-isobutyl-xanthine, dexamethasone, and insulin (MDI). See, e.g., Ntambi and CheulKim, J. Nutrition, 120: 21225-21265 for description of MDI differentiation cocktail. Results for this experiment are shown in FIG. 9.

Cells incubated with MDI, with or without infection with Ad-36, had a significant increase in FAS expression. Cells infected with Ad-36 and then incubated with MDI and 10 μM of Tridox had a modest decrease in FAS expression, but cells pretreated with Tridox and then incubated with MDI and virus had a more marked decrease in FAS expression. These data show that Tridox inhibits infection with Ad-36. Western blot of FAS protein expression in the samples showed that the amount of FAS decreased if the 3T3-L1 cells were infected with Ad-36 and then treated with 10 μM Tridox. FAS protein levels were even lower in 3T3-L1 cells exposed to Ad-36 in media containing 10 μM Tridox.

### Example 5

### Effect of Luteolin on TCID50 of A549 Cells Exposed to Ad-36

Luteolin is another antiviral that was found to inhibit Ad-36 infection. The experimental conditions were assessed: (A) Control condition in which cells were infected with the virus throughout incubation, no luteolin added; (B) Cells and virus were both pretreated with luteolin, then the cells were incubated with both the virus and luteolin; and (C) Cells pretreated with luteolin only, luteolin removed, then cells incubated with virus but without luteolin. The TCID50 value was calculated for each group.

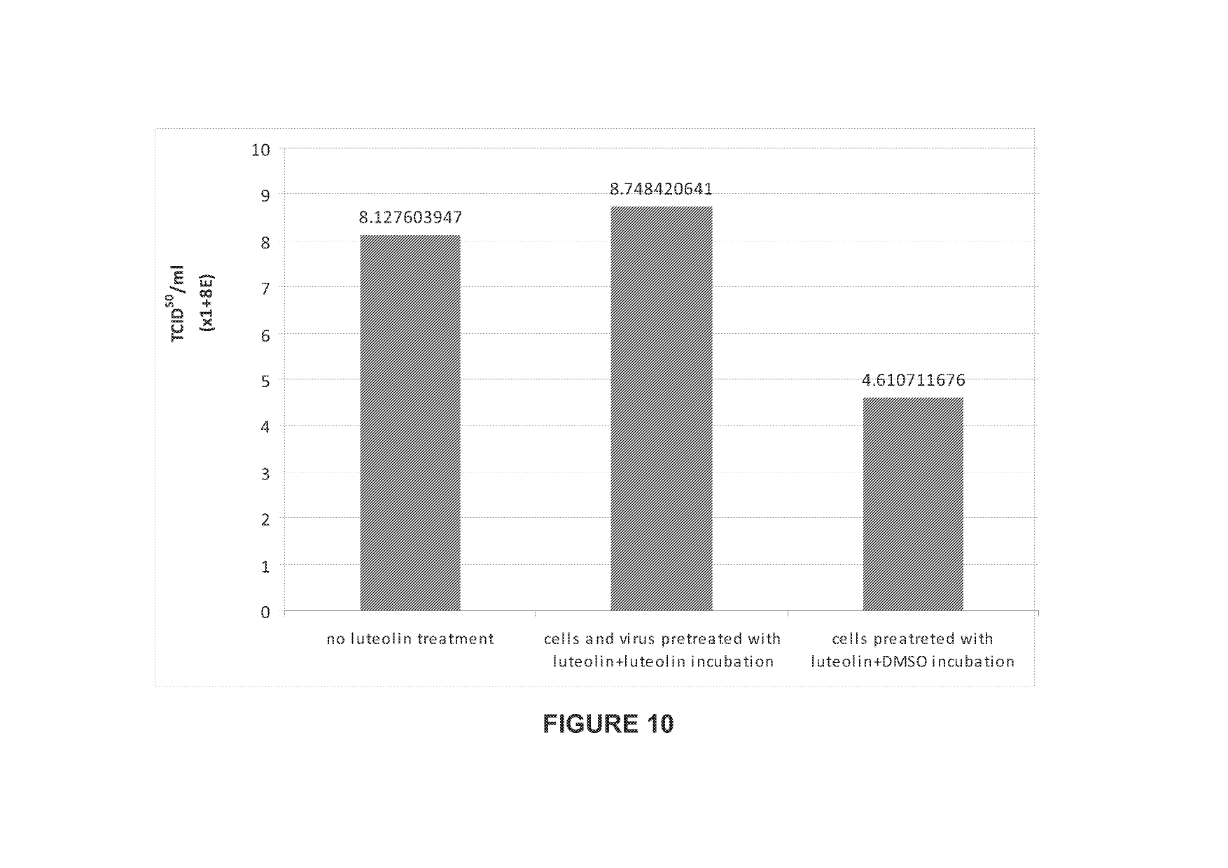

As shown in FIG. 10, luteolin blocks virulence of Ad-36 infection in A549 cells, a human lung cancer cell line. A lower value indicates decreased virulence of the virus. The TCID50 value in Group B is slightly higher than control. This is similar to a second experiment (not shown) in which the TCID value for cells under the same condition as Group B was increased almost twice as much as the control value. Luteolin kills infected cells and dead cells look similar to CPE. The higher TCID value is not caused by an increase of virulence. It is caused by increase of dead infected cells. Group C had the lowest TCID value, suggesting pre-treating cells with luteolin lowers the virulence of Ad-36. These data demonstrate that luteolin prevents cells from being infected with Ad-36.

### Example 6

### Effects of Ad-36 Infection and Luteolin on Growth Rate of MCF-10A

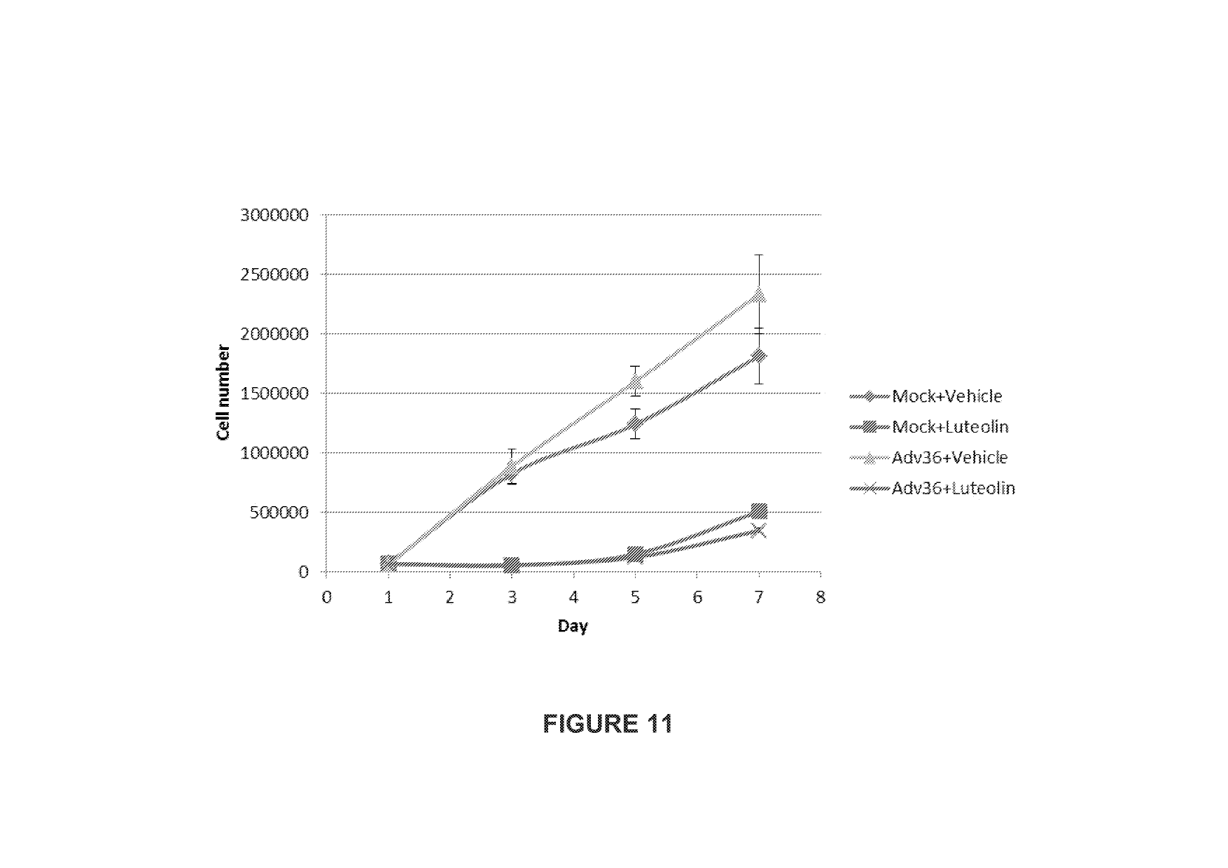

Luteolin reduces the growth rate of human breast cancer cells (MCF-10A) that have been infected with Ad-36 in tissue culture (FIG. 11). MCF-10A cells were infected with a multiplicity of infection (MOI) of 0.5 at day 0 for 2 hours, then replaced with fresh medium containing 5 μM luteolin or vehicle. At each time period, cells were trypsinzed and counted using a hemocytometer. Fresh luteolin (5 μM) was added on day 0 (after infection), day 3 and day 5 in order to insure a constant concentration of agent. FIG. 11 shows cell number after infection with Ad-36 with or without luteolin. The cells infected with Ad-36 and not exposed to luteolin had the highest growth rate, faster than cells without Ad-36. When luteolin is added, cell growth is slowed. The slowest growth rate is that of infected cells treated with luteolin, demonstrating that luteolin inhibits virus-stimulated growth rate.

### Example 7

### Effect of Luteolin and/or Metformin on Akt Phosphorylation in Ad-36-Infected MCF-10A Cells

Both luteolin and metformin and the combination of luteolin and metformin reduce levels of phosphorylated (activated) Akt, a cancer marker, in human breast cells (MCF-10A).

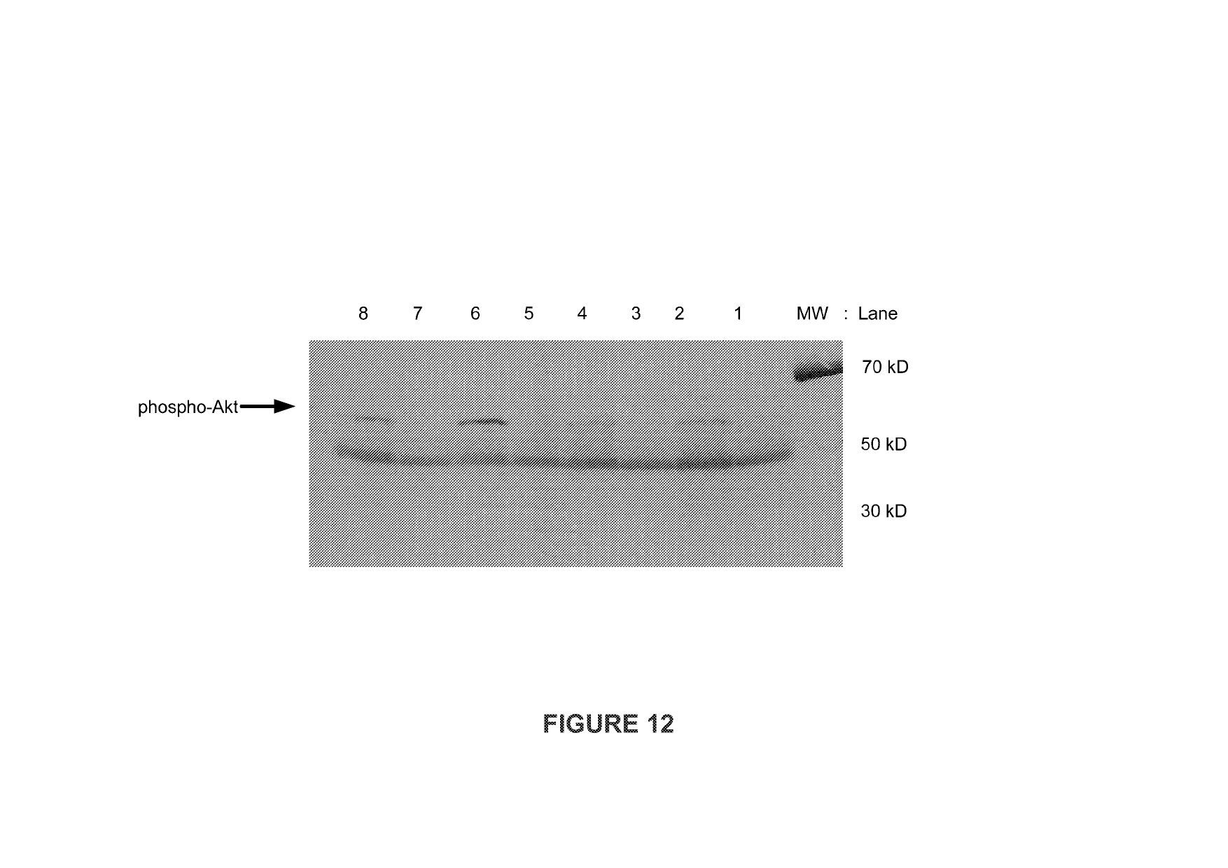

MCF-10A cells were infected with Ad-36 and treated with luteolin and/or metformin, then Akt phosphorylation was measured using Western blot. The top band is phosphorylated Akt. FIG. 12 shows eight groups: Lane 1 is MCF-10A cells alone. Lane 2 is MCF-10A cells infected with Ad-36 virus. Lane 3 is metformin, MCF-10A cells infected with Ad-36 virus. Lane 4 is uninfected MCF-10A cells treated with metformin. Lane 5 is MCF-10A cells infected with Ad-36 virus and treated with luteolin. Lane 6 is uninfected MCF-10A cells treated with luteolin. Lane 7 is MCF-10A cells infected with Ad-36 and treated with a combination of metformin and luteolin. Lane 8 is uninfected MCF-10A cells treated with a combination of metformin and luteolin. Akt phosphorylation is stimulated by virus (lane 2), luteolin alone or metformin alone (lanes 4 and 6, respectively), or the combination of luteolin and metformin (lane 8). However, there is a marked reduction in Akt phosphorylation when the cells are infected with virus and treated with luteolin alone, metformin alone, or a combination of luteolin and metformin (lanes 3, 5, and 7, respectively). Thus, these compounds can block the Akt activation that is mediated by Ad-36. This observation suggests that either or both of these drugs can reduce the aggressiveness of cancers due to Ad-36.

### Example 8

### Effect of Luteolin and/or Metformin on Replication of Ad-36 in A549 Cells

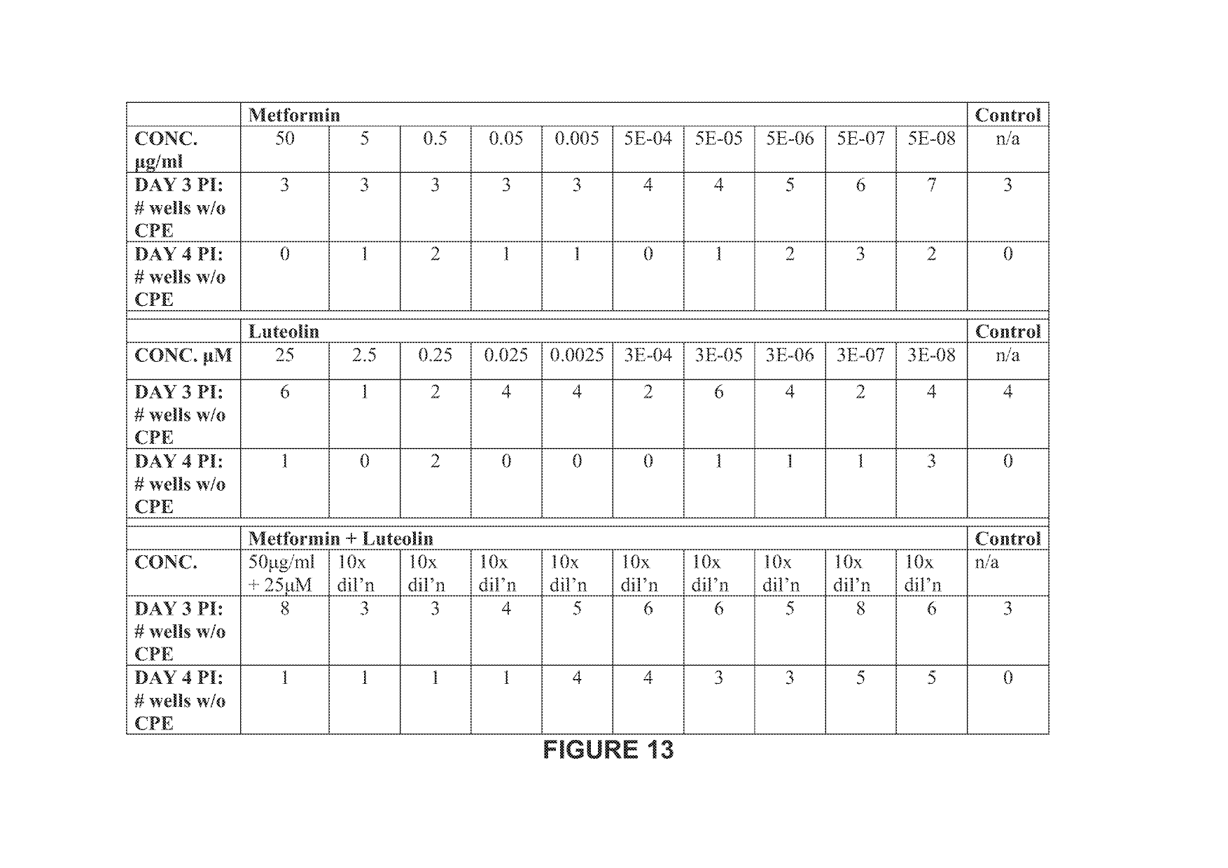

Luteolin alone, metformin alone, and the combination of luteolin and metformin in multiple different doses inhibits the effect of Ad-36 on A549 cells. This effect is due to inhibition of replication of Ad-36 due to the agents as the effect was determined by assessing cytopathic effect (CPE) which is caused by virus replication and cell death. A549 cells were infected with a fixed amount of Ad-36 (100 TCID/well) for 2 hours, then media containing the virus was removed, replaced with serial dilutions of metformin, luteolin, or a combination. Cytopathic effect (CPE) was evaluated 3 days and 4 days post-infection (PI). Each condition had 8 wells. The efficacy of the different drug treatments was evaluated by determining whether there were fewer wells with CPE as compared to the control condition for the different treatment conditions, which reflected the degree to which a particular treatment was working to inhibit the virus. The results of these experiments are shown in FIG. 13.

Viral inhibition is apparent at low doses of all of the agents, but the combination of metformin and luteolin was particularly in reducing CPE. The effect of viral inhibition by the combination drug treatment ranged from additive to synergistic. On day 4, when the effects of the single agents had diminished, the combination was still quite effect at concentrations of metformin ranging from 0.005 to 0.00000005 μg/ml and of luteolin at concentrations of 0.0025 to 0.000000025 μM. Four of the these six lowest concentrations showed a greater effect of the combination than of the additive effects of the two agents when used alone, e.g., a synergistic effect. The data show fewer CPE with the combination of metformin and luteolin present in media compared to the results with metformin or luteolin alone, especially at the lower concentrations and at day 4 post-infection. This suggests that the combination has additive and synergistic effects compared to either agent alone.

The invention has been described generally and with an emphasis on particular embodiments and aspects. The foregoing is considered as illustrative only of the principal of the invention. Because numerous modifications and changes will readily occur to those skilled in the art, the disclosure is not intended to limit the invention to the exact embodiments shown and described, and all suitable modifications and equivalents falling within the scope of the appended claims are deemed within the inventive concept. It is intended, contemplated, and therefore within the scope of the invention to combine any of the plurality of different elements in each of the aspects in the above disclosure with any other aspect of the invention. The invention is not to be limited by the disclosure above but rather is defined by the claims below. Moreover, the list of references that are mentioned in the disclosure are herein incorporated by reference in their entirety.

1. A method for treating a lipogenic adenovirus-related condition comprising:
a) identifying a subject infected with a lipogenic adenovirus and having a lipogenic adenovirus-related condition; and
b) administering a composition comprising an effective dose of an antiviral agent to the subject thereby preventing or reducing lipogenic adenovirus proliferation and/or action or thereby reducing or eliminating one or more symptoms of the lipogenic adenovirus-related condition.

2. The method of 1, further comprising administering a composition comprising an effective dose of a therapeutic agent known to treat the lipogenic adenovirus-related condition in conjunction with the composition comprising an effective dose of an antiviral agent, wherein administration of the antiviral agent and the therapeutic agent reduce or eliminate one or more symptoms of the lipogenic adenovirus-related condition more efficiently than administration of either the antiviral agent or the therapeutic agent alone.

3. The method of claim 1, wherein the step of identifying a subject having a lipogenic adenovirus-related condition comprises at least one of:
(i) determining whether a nucleic acid sequence specific to the lipogenic adenovirus is present in the biological sample;
(ii) determining whether antibodies specific to the lipogenic adenovirus are present in the biological sample; or
(iii) determining whether lipogenic adenovirus proteins and/or lipogenic adenovirus particles are present in the biological sample.

4-5. (canceled)

6. The method of claim 1, wherein the lipogenic adenovirus-related condition is selected from the group consisting of cancer, obesity, diabetes, pancreatic dysfunction, liver disease, liver dysfunction, cirrhosis, muscle dysfunction, pulmonary dysfunction, brain and nervous system dysfunction, and adrenal dysfunction.

7. (canceled)

8. The method of claim 6, wherein the cancer is one or more of prostate cancer, breast cancer, uterine cancer, ovarian cancer, colon cancer, lung cancer, kidney cancer, and pancreatic cancer.

9. The method of claim 1, wherein the lipogenic adenovirus-related condition comprises diabetes mellitus or Alzheimer's disease.

10. (canceled)

11. The method of claim 1, wherein the subject is a human mammal, a non-human mammal, or an avian animal.

12. (canceled)

13. The method of claim 1, wherein the lipogenic adenovirus comprises one or more of adenovirus type 5, adenovirus type 36, and adenovirus type 37.

14. (canceled)

15. The method of claim 1, wherein the antiviral agent comprises one or more of a ribonucleotide reductase inhibitor, a nucleoside analog, a nucleotide analog, a protease inhibitor, an antisense drug, a ribozyme, a trace mineral binder, an antioxidant, an AMP-activated protein kinase (AMPK) activator, and/or an interferon drug.

16-17. (canceled)

18. The method of claim 2, wherein the therapeutic agent comprises a chemotherapeutic agent.

19. The method of claim 2, wherein the therapeutic agent comprises one or more of an alkylating agent, an antimetabolite, an anthracycline, a plant alkaloid, a topoisomerase inhibitor, a cytotoxic antibiotic, a targeted therapeutic, a hormone, or an antihyperglycemic drug.

20. (canceled)

21. The method of claim 1, wherein the antiviral agent inhibits and/or reduces expression of lipogenic enzymes and/or lipogenic transcription factors.

22. The method of claim 21, wherein the lipogenic enzymes comprise one or more of fatty acid synthase (FAS), glycerol-3-phosphophate dehydrogenase (GPDH), lipoprotein lipase (LPL), stearoyl-CoA desaturase 1 (SCD1), carnitine palmitoyltransferase 1 (CPT1), L-type pyruvate kinase (L-PK), proteins in the phosphatidylinositol 3-kinase (PI3K) signaling pathway, and proteins in the AKT/Protein Kinase B (PKB) signaling pathway.

23. The method of claim 1, wherein the antiviral agent inhibits and/or reduces the expression of adipocyte differentiation factors.

24. The method of claim 23, wherein the adipocyte differentiation factors comprise one or more of peroxisome proliferator-activated receptor gamma (PPAR-γ), CCAAT/enhancer binding protein alpha (C/EBP-α), C/EBP-β, sterol regulatory element-binding protein 1 (SREBP-1), and carbohydrate responsive element- binding protein (ChREBP).

25-26. (canceled)

27. The method of claim 6, wherein the prevention or reduction of lipogenic adenovirus proliferation reduces cancer aggressiveness.

28. A therapeutic composition for use in reducing or eliminating one or more symptoms of a lipogenic adenovirus-related condition comprising
a) an effective dose of an antiviral agent, wherein administration of the antiviral agent prevents or reduces lipogenic adenovirus proliferation and/or action;
b) an effective dose of a therapeutic agent, wherein the therapeutic agent known to treat the lipogenic adenovirus-related condition; and
c) a pharmaceutically acceptable carrier.

29-58. (canceled)

59. The method of claim 1, further comprising administering Didox, Tridox or luteolin to the subject thereby reducing fatty acid synthase (FAS) expression, AKT phosphorylation, and or viral replication.

60. A method of reducing fatty acid synthase (FAS) expression in a subject infected with a lipogenic adenovirus comprising:
a) identifying a subject infected with a lipogenic adenovirus; and
b) administering to the subject an effective dose of an antiviral agent.

61. The method of claim 60, wherein the antiviral agent comprises Didox, Tridox, luteolin, or metformin.
